(19) United States
(12) Patent Application Publication
(10) Pub. No.: US 2024/0255847 A1
(43) Pub. Date: Aug. 1, 2024

(54) ORGANIC SALT, PHOTORESIST COMPOSITION INCLUDING THE SAME, AND METHOD OF FORMING PATTERN BY USING THE SAME

(71) Applicant: Samsung Electronics Co., Ltd., Suwon-si (KR)

(72) Inventors: Hana KIM, Suwon-si (KR); Yoonhyun KWAK, Suwon-si (KR); Hyeran KIM, Suwon-si (KR); Beomseok KIM, Suwon-si (KR); Hoyoon PARK, Suwon-si (KR); Sunyoung LEE, Suwon-si (KR); Minyoung HA, Suwon-si (KR)

(73) Assignee: Samsung Electronics Co., Ltd., Suwon-si (KR)

(21) Appl. No.: 18/404,865

(22) Filed: Jan. 4, 2024

(30) Foreign Application Priority Data
Jan. 6, 2023 (KR) 10-2023-0002501

(51) Int. Cl. G03F 7/004; G03F 7/039

(52) U.S. Cl. CPC G03F 7/0045; G03F 7/0048; G03F 7/039

(57) ABSTRACT

Provided are an organic salt represented by Formula 1 below, a photoresist composition including the same, and a method of forming a pattern by using the photoresist composition:

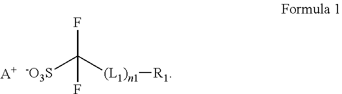

For descriptions of L₁, n1, R₁, and A⁺ in Formula 1, refer to those provided herein.

### CROSS-REFERENCE TO RELATED APPLICATION

This application is based on and claims priority under 35 U.S.C. § 119 to Korean Patent Application No. 10-2023-0002501, filed on Jan. 6, 2023, in the Korean Intellectual Property Office, the disclosure of which is incorporated by reference herein in its entirety.

### BACKGROUND

### 1. Field

Provided are an organic salt, a photoresist composition including the same, and a method of forming a pattern by using the composition.

### 2. Description of the Related Art

In semiconductor manufacturing, photoresists may have physical properties that change in response to light and photoresists may be used to form fine patterns. Among the photoresists, chemically amplified photoresists have been widely used. In the chemically amplified photoresists, an acid formed by reacting light with a photoacid generator reacts again with a base resin to change solubility of the base resin in a developing solution, and thus, patterning is made possible.

In particular, when using high-energy rays with relatively high energy, such as extreme ultraviolet (EUV) rays, the number of photons may be significantly small even when light of the same energy is irradiated. Accordingly, there may be a demand for a photoacid generator that may be effective even when a small amount is used and that provides improved sensitivity and/or resolution.

In addition, for chemically amplified photoresists, as the formed acid diffuses to an unexposed area, issues such as reduced uniformity of a pattern or increased surface roughness may occur, and thus, there may be a demand for a quencher with improved dispersibility and/or improved compatibility with a base resin.

### SUMMARY

Provided are an organic salt that may act as a photoacid generator capable of providing improved sensitivity and/or resolution, a photoresist composition including the same, and a method of forming a pattern by using the composition.

Additional aspects will be set forth in part in the description which follows and, in part, will be apparent from the description, or may be learned by practice of the presented embodiments of the disclosure.

According to an example embodiment, an organic salt may be represented by Formula 1 below:

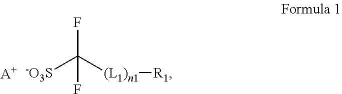

wherein, in Formula 1,
L1 may be a single bond or a divalent linking group,
L1 may not contain oxygen (O),
n1 may be an integer of 1 to 5,
R₁ may be a cyclic monovalent C1-C20 hydrocarbon group, which may optionally contain a heteroatom, and
R₁ may not include a group represented by the following Formula R1 or R2,

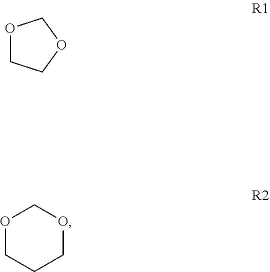 [SMILES: C1COCO1 ; C1COCOC1]

and
A⁺ may be a counter cation.

According to an example embodiment, a photoresist composition may include the organic salt, an organic solvent, and a base resin.

According to an example embodiment, a method of forming a pattern may include: applying the above-described photoresist composition to form a photoresist film on a substrate; exposing at least a portion of the photoresist film with high-energy rays to provide an exposed photoresist film; and developing the exposed photoresist film using a developing solution.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a flowchart illustrating a method of forming a pattern, according to an embodiment of the present disclosure;

FIGS. 2A to 2C are side cross-sectional views showing a method of forming a pattern, according to an embodiment of the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list. For example, “at least one of A, B, and C,” and similar language (e.g., “at least one selected from the group consisting of A, B, and C”) may be construed as A only, B only, C only, or any combination of two or more of A, B, and C, such as, for instance, ABC, AB, BC, and AC.

When the terms “about” or “substantially” are used in this specification in connection with a numerical value, it is intended that the associated numerical value includes a manufacturing or operational tolerance (e.g., ±10%) around the stated numerical value. Moreover, when the words “generally” and “substantially” are used in connection with geometric shapes, it is intended that precision of the geometric shape is not required but that latitude for the shape is within the scope of the disclosure. Further, regardless of whether numerical values or shapes are modified as “about” or “substantially,” it will be understood that these values and shapes should be construed as including a manufacturing or operational tolerance (e.g., ±10%) around the stated numerical values or shapes. When ranges are specified, the range includes all values therebetween such as increments of 0.1%.

The present disclosure may be modified in various ways and have many embodiments, and particular embodiments are illustrated in the drawings and are described in the detailed description. However, this does not intend to limit the present disclosure within particular embodiments, and it should be understood that the present disclosure covers all the modifications, equivalents, and replacements within the idea and technical scope of the present disclosure. In describing the present disclosure, detailed descriptions of related known art will be omitted when it is determined that the detailed descriptions may obscure the gist of the present disclosure.

It will be understood that, although the terms “first”, “second”, “third”, etc., may be used herein to describe various elements, these terms are only used to distinguish one element from another element, and the order, type, etc. of the elements are not limited.

As used herein, when an element such as a layer, a film, a region, or a substrate is referred to as being “on” or “above” another element, it may be right above, below, left, or right in contact as well as being above, below, left, or right without contact.

The singular of any term includes the plural, unless the context otherwise requires. As used herein, the expression of “include” or “have” indicates a presence of a characteristic, a number, a phase, a movement, an element, a component, or combinations of components described in the specification, and it should not be construed to exclude in advance an existence or possibility of existence of at least one of other characteristics, numbers, movements, elements, components, or combinations of components.

Wherever a range of values is given, that range includes every value falling within the range, as if written out explicitly, and further includes the values bounding the range. Thus, a range of “X to Y” includes every value falling between X and Y, and also includes X and Y.

In the present specification, “Cₓ-C_{y}” means that a number of carbons constituting a substituent is x to y. For example, “C₁-C₆” means that a number of carbons constituting the substituent is 1 to 6, and “C₆-C₂₀” means that a number of carbons constituting the substituent is 6 to 20.

In this specification, “monovalent hydrocarbon group” may include, for example, a linear or branched alkyl group (for example, a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, and a nonyl group); a monovalent saturated cycloaliphatic hydrocarbon group (for example, a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a 1-adamantyl group, a 2-adamantyl group, a 1-adamantylmethyl group, norbornyl group, a norbornylmethyl group, a tricyclodecanyl group, a tetracyclododecanyl group, a tetracyclododecanylmethyl group, and a dicyclohexylmethyl group); a monovalent unsaturated aliphatic hydrocarbon group (for example, an allyl group, and a 3-cyclohexenyl group); an aryl group (for example, a phenyl group, a 1-naphthyl group, and a 2-naphthyl group); an arylalkyl group (for example, a benzyl group, and a diphenylmethyl group); and a monovalent hydrocarbon group containing a heteroatom (for example, a tetrahydrofuranyl group, a methoxymethyl group, an ethoxymethyl group, a methylthiomethyl group, an acetamidemethyl group, a trifluoroethyl group, (2-a methoxyethoxy)methyl group, an acetoxymethyl group, a 2-carboxy-1-cyclohexyl group, a 2-oxopropyl group, a 4-oxo-1-adamantyl group, and a 3-oxocyclohexyl group). In addition, in these groups, some hydrogen atoms may be substituted with a moiety containing a heteroatom such as oxygen, sulfur, nitrogen, or a halogen atom, or some carbon atoms may be replaced with a moiety containing a heteroatom such as oxygen, sulfur, or nitrogen, and thus, these groups may contain a hydroxyl group, a cyano group, a carbonyl group, a carboxyl group, an ether bond, an ester bond, a sulfonic acid ester bond, a carbonate, a lactone ring, a sultone ring, a carboxylic acid anhydride moiety, or a haloalkyl moiety.

In the present specification, “alkyl group” refers to a linear or branched saturated aliphatic hydrocarbon monovalent group, and specific examples include a methyl group, an ethyl group, a propyl group, an isobutyl group, a sec-butyl group, a tert-butyl group, a pentyl group, an iso-amyl group, a hexyl group, and the like. In the specification, “alkylene group” refers to a linear or branched saturated aliphatic hydrocarbon divalent group, and specific examples include a methylene group, an ethylene group, a propylene group, a butylene group, an isobutylene group, and the like.

In the present specification, “halogenated alkyl group” refers to a group in which one or more substituents of the alkyl group are substituted with halogens, and specific examples include CF₃, and the like. Here, halogens may be F, Cl, Br or I.

In the specification, “alkoxy group” refers to a monovalent group having a chemical formula of —OA₁₀₁, wherein A₁₀₁ is an alkyl group. Specific examples thereof include a methoxy group, an ethoxy group, an isopropyloxy group, and the like.

In the present specification, “cycloalkyl group” refers to a monovalent saturated hydrocarbon cyclic group, and specific examples thereof include a monocyclic group such as a cyclopropyl group, a cyclobutyl group, a cyclopentyl group, a cyclohexyl group, a cycloheptyl group, and the like, and a polycyclic condensed cyclic group such as a norbornyl group, and an adamantyl group. In the present specification, “cycloalkylene group” refers to a divalent saturated hydrocarbon cyclic group, and specific examples thereof include a cyclopentylene group, a cyclohexylene group, an adamantylene group, an adamantylmethylene group, a norbornylene group, a norbornylmethylene group, a tricyclodecanylene group, a tetracyclododecanylene group, a tetracyclododecanylmethylene group, a dicyclohexylmethylene group, and the like.

In the specification, “cycloalkoxy group” refers to a monovalent group having a chemical formula of —OA₁₀₂, wherein A₁₀₂ is a cycloalkyl group. Specific examples thereof include a cyclopropoxy group, a cyclobutoxy group, and the like.

In the present specification, “heterocycloalkyl group” may be one in which some carbon atoms of the cycloalkyl group are replaced with a moiety containing a heteroatom, such as oxygen, sulfur, or nitrogen. The heterocycloalkyl group may include an ether bond, an ester bond, a sulfonic acid ester bond, carbonate, a lactone ring, a sultone ring, or a carboxylic acid anhydride moiety. In the present specification, a “heterocycloalkylene group” is one in which some carbon atoms of the cycloalkylene group are replaced with a moiety containing a heteroatom, such as oxygen, sulfur, or nitrogen.

In the present specification, “alkenylene group” refers to a linear or branched unsaturated aliphatic hydrocarbon divalent group.

In the present specification, “cycloalkenylene group” refers to a divalent unsaturated hydrocarbon cyclic group.

In the present specification, “heterocycloalkenylene group” refers to one in which some carbon atoms of the cycloalkenylene group are replaced with a moiety including a heteroatom, such as oxygen, sulfur, or nitrogen.

In the present specification, “aryl group” refers to a monovalent group having a carbocyclic aromatic system, and specific examples thereof include a phenyl group, a naphthyl group, an anthracenyl group, a phenanthrenyl group, a pyrenyl group, a chrysenyl group, and the like. In the present specification, “arylene group” refers to a divalent group having a carbocyclic aromatic system.

In the present specification, “heteroaryl group” refers to a monovalent group having a heterocyclic aromatic system, and specific examples include a pyridinyl group, a pyrimidinyl group, a pyrazinyl group, and the like. In the present specification, “heteroarylene group” refers to a divalent group having a heterocyclic aromatic system.

In the present specification, “cycloalkyl group” refers to a monovalent, divalent, or trivalent group in which any hydrogen of the cycloalkyl group may be further substituted with a binding site.

In the present specification, “heterocycloalkyl group” refers to a monovalent, divalent, or trivalent group in which any hydrogen of the heterocycloalkyl group may be further substituted with a binding site.

In the present specification, “cycloalkenyl group” refers to a monovalent, divalent, or trivalent group in which any hydrogen of the cycloalkenyl group may be further substituted with a binding site.

In the present specification, “heterocycloalkenyl group” refers to a monovalent, divalent, or trivalent group in which any hydrogen of the heterocycloalkenyl group may be further substituted with a binding site.

In the present specification, “aryl group” refers to a monovalent, divalent, or trivalent group in which any hydrogen of the aryl group may be further substituted with a binding site.

In the present specification, “heteroaryl group” refers to a monovalent, divalent, or trivalent group in which any hydrogen of the heteroaryl group may be further substituted with a binding site.

In the present specification, “counter cation” refers to any cation capable of binding by ionic bonding with an anion, and generally refers to a cation that limits and/or prevents a compound from being charged.

Hereinafter, embodiments of the present disclosure are described in detail with reference to the accompanying drawings, and when describing the present disclosure with reference to the drawings, substantially identical or corresponding components are given the same reference numerals, and redundant descriptions will be omitted. In the drawings, thicknesses are shown enlarged to clearly express various layers and regions. In addition, in the drawings, the thicknesses of some layers and regions are exaggerated for convenience of description. Meanwhile, the embodiments described below are merely exemplary, and various modifications are possible from these embodiments.

### [Organic Salt]

An organic salt according to an embodiment is represented by Formula 1 below:

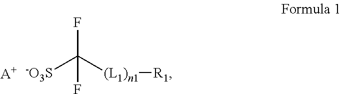

wherein, in Formula 1,
L₁ may be a single bond or a divalent linking group,
L₁ may not contain oxygen (O), n1 may be an integer of 1 to 5,
R₁ may be a cyclic monovalent C₁-C₂₀ hydrocarbon group, which may optionally contain a heteroatom, and
R₁ may not include a group represented by the following Formula R1 or R2,

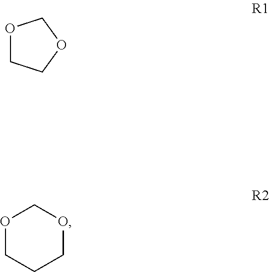 [SMILES: C1COCO1 ; C1COCOC1]

and
A⁺ may be a counter cation.

For example, L₁ in Formula 1 may be a single bond, S, or a linear, branched, or cyclic divalent C₁-C₂₀ hydrocarbon group.

In some embodiments, L₁ in Formula 1 may be a single bond; S; or a C₁-C₂₀ alkylene group, a C₂-C₂₀ alkenylene group, a C₃-C₂₀ cycloalkenylene group, a C₃-C₂₀ heterocycloalkenylene group, a C₆-C₂₀ arylene group, or a C₁-C₂₀ heteroarylene group, unsubstituted or substituted with deuterium, a halogen, a C₁-C₂₀ alkyl group, a C₃-C₂₀ cycloalkyl group, a C₁-C₂₀ heterocycloalkyl group, a C₆-C₂₀ aryl group, a C₁-C₂₀ heteroaryl group, or any combination thereof.

In some embodiments, L₁ in Formula 1 may be a single bond; or a C₁-C₂₀ alkylene group unsubstituted or substituted with deuterium, a halogen, a C₁-C₂₀ alkyl group, or any combination thereof.

For example, R₁ in Formula 1 may be a cyclic monovalent C₁-C₂₀ hydrocarbon group that may optionally contain a heteroatom, wherein the heteroatom may not contain oxygen (O).

For example, R₁ in Formula 1 may be a C₃-C₂₀ cycloalkyl group, a C₁-C₂₀ heterocycloalkyl group, a C₆-C₂₀ aryl group, or a C₃-C₂₀ heteroaryl group, unsubstituted or substituted with deuterium, a halogen, a C₁-C₂₀ alkyl group, a C₃-C₂₀ cycloalkyl group, a C₆-C₂₀ aryl group, a C₃-C₂₀ heteroaryl group, or any combination thereof.

In some embodiments, R₁ in Formula 1 may be a cyclohexyl group, an adamantyl group, a norbornyl group, a tricyclodecanyl group, a tetracyclododecanyl group, a phenyl group, a naphthyl group, or a pyridinyl group, unsubstituted or substituted with deuterium, a halogen, a C₁-C₂₀ alkyl group, a C₃-C₂₀ cycloalkyl group, a C₆-C₂₀ aryl group, a C₃-C₂₀ heteroaryl group, or any combination thereof.

In some embodiments, R₁ in Formula 1 may be a cyclohexyl group, an adamantyl group, a norbornyl group, a tricyclodecanyl group, a tetracyclododecanyl group, a phenyl group, a naphthyl group, or a pyridinyl group, unsubstituted or substituted with —F, —Cl, —Br, —I, a C₁-C₁₀ alkyl group, a cyclopentyl group, a cyclohexyl group, a phenyl group, a naphthyl group, or any combination thereof.

For example, A⁺ in Formula 1 may be a substituted or unsubstituted sulfonium cation, a substituted or unsubstituted iodonium cation, or any combination thereof.

In some embodiments, A⁺ in Formula 1 may be represented by Formula 2-1 or 2-2:

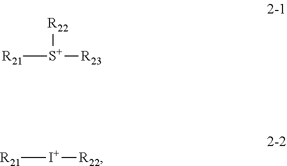

wherein in Formulas 2-1 and 2-2,
R₂₁ to R₂₃ are each independently a linear, branched, or cyclic monovalent C₁-C₃₀ hydrocarbon group which may optionally contain a heteroatom, and
two adjacent groups of R₂₁ to R₂₃ may be optionally bound to each other to form a ring.

In some embodiments, A⁺ in Formula 1 may be represented by Formula 2-11 or 2-12:

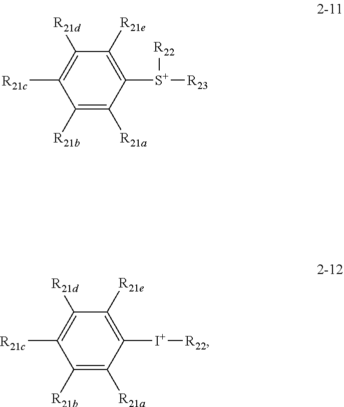

wherein in Formulas 2-11 and 2-12,
R₂₁ₐ to R₂₁ₑ may each independently be a linear, branched, or cyclic monovalent C₁-C₂₀ hydrocarbon group which may optionally contain hydrogen, deuterium, a halogen, a cyano group, a hydroxyl group, or a heteroatom,
R₂₂ to R₂₃ are each independently a linear, branched, or cyclic monovalent C₁-C₃₀ hydrocarbon group which may optionally contain a heteroatom, and
two adjacent groups of R₂₁ₐ to R₂₁ₑ, and R₂₂ to R₂₃ may be optionally bound to each other to form a ring.

In some embodiments, A⁺ in Formula 1 may be represented by any one of the following Formulas 2-21 to 2-23:

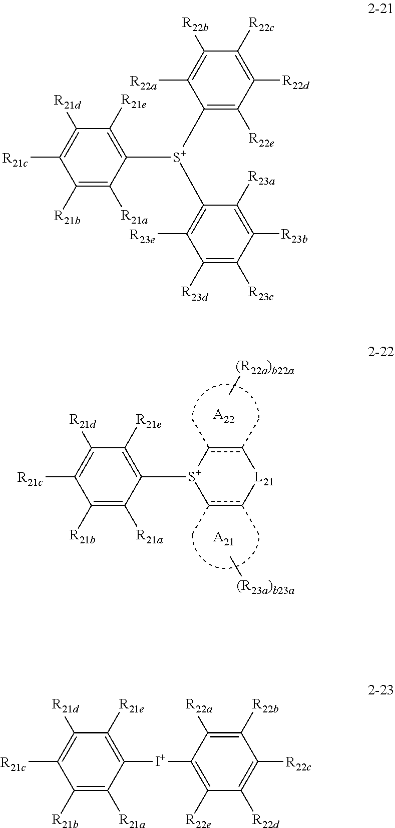

wherein in Formulas 2-21 to 2-23,
R₂₁ₐ to R₂₁ₑ, R₂₂ₐ to R₂₂ₑ, and R₂₃ₐ to R₂₃ₑ may each independently be a linear, branched, or cyclic monovalent C₁-C₂₀ hydrocarbon group which may optionally contain hydrogen, deuterium, a halogen, a cyano group, a hydroxyl group, or a heteroatom,
two adjacent groups of R₂₁ₐ to R₂₁ₑ, R₂₂ₐ to R₂₃ₑ, and R₂₃ₐ to R₂₃ₑ may be optionally bound to each other to form a ring,
b22a and b23a may be each an integer of 1 to 4,
A₂₁ and A₂₂ may each independently be absent, or a benzene ring,
each [symbol] may be a carbon-carbon single bond or a carbon-carbon double bond,
L₂₁ may be a single bond, O, S, CO, SO, SO₂, CRR′, or NR, and

R and R′ may each independently be a linear, branched, or cyclic monovalent C₁-C₂₀ hydrocarbon group, which may optionally contain hydrogen, deuterium, a halogen, a cyano group, a hydroxyl group, or a heteroatom.

In an embodiment, A⁺ in Formula 1 may be selected from Group I below:

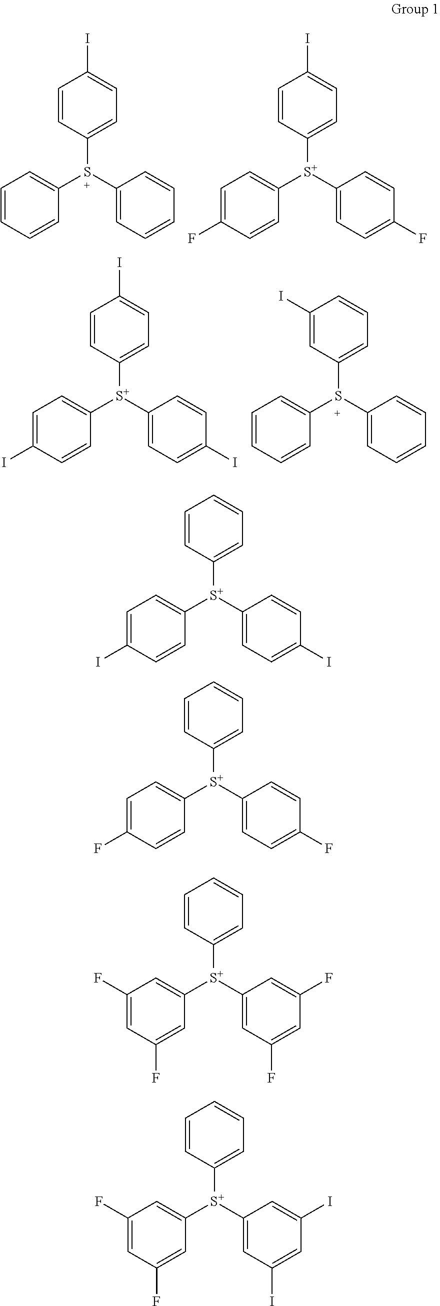

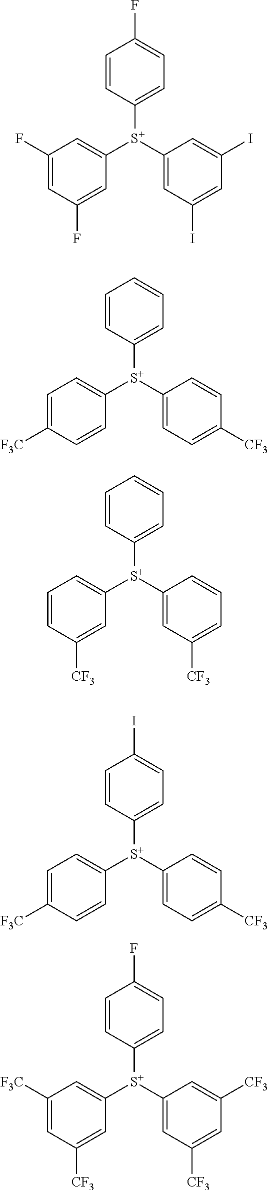 [SMILES: Fc1ccc([S+](c2cc(F)cc(F)c2)c2cc(I)cc(I)c2)cc1 ; FC(F)(F)c1ccc([S+](c2ccccc2)c2ccc(C(F)(F)F)cc2)cc1 ; FC(F)(F)c1cccc([S+](c2ccccc2)c2cccc(C(F)(F)F)c2)c1 ; FC(F)(F)c1ccc([S+](c2ccc(I)cc2)c2ccc(C(F)(F)F)cc2)cc1 ; Fc1ccc([S+](c2cc(C(F)(F)F)cc(C(F)(F)F)c2)c2cc(C(F)(F)F)cc(C(F)(F)F)c2)cc1]

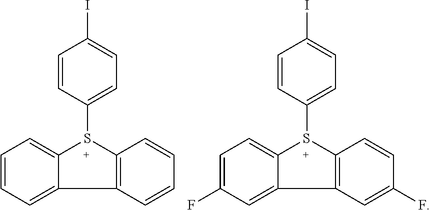

In an embodiment, an organic salt represented by Formula 1 may be represented by Formula 1-1 below:

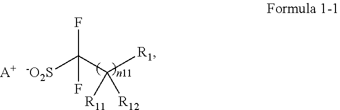

wherein in Formula 1-1,
descriptions of R₁ and A⁺ are the same as described herein, respectively,
R₁₁ and R₁₂ may each independently be hydrogen, a halogen, a C₁-C₂₀ alkyl group, or a C₁-C₂₀ halogenated alkyl group, and
n11 may be an integer of 0 to 5.

In some embodiments, the organic salt represented by Formula 1 may be selected from Group II below:

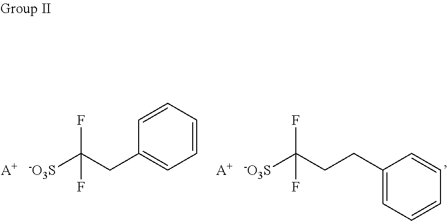

wherein, in Group II, A⁺ may be a counter cation.

In some embodiments, A⁺ in Group II may be selected from Group I.

Typically, since EUV (13.5 nm) has a lower number of photons compared to an ArF immersion light source, as an exposure dose decreases, noise increases more significantly in the boundary area between an area exposed by the EUV light source and an unexposed area that is not exposed. For a lithography process using an EUV light source, in order to compensate for the noise, a larger amount of a photoacid generator must be used compared to a lithography process using another light source of the same amount of light. However, when a photoresist composition includes a high content of a photoacid generator, a glass transition temperature (Tg) of the base resin may change and thermal stability may deteriorate. In addition, resolution of formed resist patterns may be deteriorated due to a photoacid generator remaining during a lithography process using an EUV light source.

Since the organic salt represented by Formula 1 does not include oxygen (O) as a linker (L1 in Formula 1), the organic salt may exhibit superior etching resistance compared to organic salts including oxygen as a linker.

In addition, since the organic salt represented by Formula 1 exhibits an improved acid generating effect, the photoresist composition, including the organic salt represented by Formula 1, may provide a pattern with improved resolution when forming a pattern by using an EUV light source, even when a relatively small amount (e.g., the same or smaller amount) than when using a light source other than EUV, is used.

### Photoresist Composition

According to another aspect, provided is a photoresist composition including the organic salt, an organic solvent, and a base resin. The photoresist composition may have properties such as improved developability and/or improved resolution.

The photoresist composition changes solubility in a developing solution by exposure to high-energy rays. The photoresist composition may be a positive-type photoresist composition, in which an exposed portion of the photoresist film is dissolved and removed to form a positive-type photoresist pattern, or a negative photoresist composition in which a non-exposed portion of the photoresist film is dissolved and removed to form a negative photoresist pattern. In addition, a sensitive photoresist composition according to an embodiment may be for an alkali developing process using an alkali developing solution for a developing process in forming a photoresist pattern, or for a solvent developing process using a developing solution (hereinafter referred to as an organic developing solution) including an organic solvent for the developing process.

The organic salt may be a photo-decomposable compound that may be decomposed by exposure to light. When the organic salt is decomposed by exposure and generates an acid, the organic salt may act as a photoacid generator, and thus, the photoresist composition may not include a separate photoacid generator. Instead of a photoacid generator, the photoresist composition may further include a quencher.

The organic salt may be used in an amount of about 0.1 parts by weight to about 80 parts by weight, about 5 parts by weight to about 60 parts by weight, with respect to 100 parts by weight of the base resin. When the above-mentioned range is satisfied, the function of a photoacid generator is exhibited at an appropriate level, and any performance loss, for example, formation of foreign particles due to a decrease in sensitivity, and/or lack of solubility may be reduced.

Since the organic salt is as described above, hereinafter, an organic solvent, a base resin, and optional components such as a photoacid generator included as necessary will be described. In addition, in the photoresist composition, one type of an organic salt may be used, or two or more different types may be used in combination.

### Organic Solvent>

The organic solvent included in the photoresist composition is not particularly limited as long as it is capable of dissolving or dispersing organic salts, base resins, photoacid generators, and optional components contained as needed. For the organic solvent, one type may be used, or two or more different types may be used in combination. In addition, a mixed solvent in which water and an organic solvent are mixed may be used.

Examples of the organic solvent include, for example, an alcohol-based solvent, an ether-based solvent, a ketone-based solvent, an amide-based solvent, an ester-based solvent, a sulfoxide-based solvent, a hydrocarbon-based solvent, and the like.

In some embodiments, the alcohol-based solvent include, for example, a monoalcoholic-based solvent such as methanol, ethanol, n-propanol, isopropanol, 1-methoxy-2-propanol, 1-ethoxy-2-propanol, n-butanol, isobutanol, sec-butanol, tert-butanol, n-pentanol, isopentanol, 2-methylbutanol, sec-pentanol, tert-pentanol, 3-methoxybutanol, 3-methyl-3-methoxybutanol, n-hexanol, 2-methylpentanol, sec-hexanol, 2-ethylbutanol, sec-heptanol, 3-heptanol, n-octanol, 2-ethylhexanol, sec-octanol, n-nonylalcohol, 2,6-dimethyl-4-heptanol, n-decanol, sec-undecyl alcohol, trimethylnonyl alcohol, sec-tetradecyl alcohol, sec-heptadecyl alcohol, furfuryl alcohol, phenol, cyclohexanol, methylcyclohexanol, 3,3,5-trimethylcyclohexanol, benzyl alcohol, diacetone alcohol, etc.; a polyhydric alcohol-based solvent such as ethylene glycol, 1,2-propylene glycol, 1,3-butylene glycol, 2,4-pentanediol, 2-methyl-2,4-pentanediol, 2,5-hexanediol, 2,4-heptanediol, 2-ethyl-1,3-hexanediol, diethylene glycol, dipropylene glycol, triethylene glycol, tripropylene glycol, etc.; and a polyhydric alcohol-containing ether-based solvent such as ethylene glycol monomethyl ether, ethylene glycol monoethyl ether, ethylene glycol monopropyl ether, ethylene glycol monobutyl ether, ethylene glycol monohexyl ether, ethylene glycol monophenyl ether, ethylene glycol mono-2-ethylbutyl ether, diethylene glycol monomethyl ether, diethylene glycol monoethyl ether, diethylene glycol monopropyl ether, diethylene glycol monobutyl ether, diethylene glycol monohexyl ether, diethylene glycol dimethyl ether, propylene glycol monomethyl ether, propylene glycol dimethyl ether, propylene glycol monoethyl ether, propylene glycol monopropyl ether, propylene glycol monobutyl ether, dipropylene glycol monomethyl ether, dipropylene glycol monoethyl ether, dipropylene glycol monopropyl ether, etc.

Examples of the ether-based solvent include a dialkyl ether-based solvent such as diethyl ether, dipropyl ether, dibutyl ether, etc.; a cyclic ether-based solvent such as tetrahydrofuran, tetrahydropyran, etc.; and an aromatic ring-containing ether solvent such as diphenyl ether, anisole, etc.

The ketone-based solvent includes, for example, a chain ketone-based solvent, such as acetone, methyl ethyl ketone, methyl-n-propyl ketone, methyl-n-butyl ketone, methyl-n-pentyl ketone, diethyl ketone, methyl isobutyl ketone, 2-heptanone, ethyl-n-butyl ketone, methyl-n-hexyl ketone, diisobutyl ketone, trimethyl nonanone, etc.; and a cyclic ketone-based solvent such as cyclopentanone, cyclohexanone, cycloheptanone, 16 yclooctenone, and methylcyclohexanone; 2,4-pentanedione, acetonylacetone, acetphenone, etc.

The amide-based solvent includes, for example, a cyclic amide-based solvent such as N,N′-dimethylimidazolidinone, and N-methyl-2-pyrrolidone; a chain amide-based solvent such as N-methylformamide, N,N-dimethylformamide, N,N-diethylformamide, acetamide, N-methylacetamide, N,N-dimethylacetamide, and N-methylpropionamide, etc.

The ester-based solvent includes, for example, an acetate ester-based solvent such as methyl acetate, ethyl acetate, n-propyl acetate, isopropyl acetate, n-butyl acetate, isobutyl acetate, sec-butyl acetate, t-butyl acetate, n-pentyl acetate, isopentyl acetate, sec-pentyl acetate, 3-methoxybutyl acetate, methylpentyl acetate, 2-ethylbutyl acetate, 2-ethylhexyl acetate, benzyl acetate, cyclohexyl acetate, methylcyclohexyl acetate, n-nonyl acetate, etc.; a polyhydric alcohol-containing ether carboxylate-based solvent such as ethylene glycol monomethyl ether acetate, ethylene glycol monoethyl ether acetate, diethylene glycol monomethyl ether acetate, diethylene glycol monoethyl ether acetate, diethylene glycol mono-n-butyl ether acetate, propylene glycol monomethyl ether acetate, propylene glycol monoethyl ether acetate, propylene glycol monopropylether acetate, propylene glycol monobutylether acetate, dipropylene glycol monomethylether acetate, dipropylene glycol monoethylether acetate, etc.; a lactone-based solvent such as γ-butyrolactone, δ-valerolactone, etc.; a carbonate-based solvent such as dimethyl carbonate, diethyl carbonate, ethylene carbonate, propylene carbonate, etc.; a lactate ester-based solvent such as methyl lactate, ethyl lactate, n-butyl lactate, n-amyl lactate, etc.; glycoldiacetate, methoxytriglycol acetate, ethyl propionate, n-butyl propionate, isoamyl propionate, diethyloxalate, di-n-butyloxalate, methyl acetoacetate, ethyl acetoacetate, diethyl malonate, dimethyl phthalate, diethyl phthalate, etc.

The sulfoxide-based solvent includes, for example, dimethyl sulfoxide, diethyl sulfoxide, etc.

The hydrocarbon-based solvent include, for example, an aliphatic hydrocarbon-based solvent such as n-pentane, isopentane, n-hexane, isohexane, n-heptane, isoheptane, 2,2,4-trimethylpentane, n-octane, isooctane, cyclohexane, methylcyclohexane, etc.; an aromatic hydrocarbon-based solvent such as benzene, toluene, xylene, mesitylene, ethylbenzene, trimethylbenzene, methylethylbenzene, n-propylbenzene, isopropylbenzene, diethylbenzene, isobutylbenzene, triethylbenzene, diisopropylbenzene, n-amylnaphthalene, etc.

In some embodiments, the organic solvent may be selected from an alcohol-based solvent, an amide-based solvent, an ester-based solvent, a sulfoxide-based solvent, and any combination thereof. In some embodiments, the solvent may be selected from propylene glycol monomethyl ether, propylene glycol monoethyl ether, propylene glycol monomethyl ether acetate, N-methyl-2-pyrrolidone, N,N-dimethylacetamide, ethyl lactate, dimethyl sulfoxide, and any combination thereof.

On the other hand, when an acid labile group in a form of acetal is used, in order to accelerate deprotection reactions of the acetal, alcohols with high boiling points, such as diethylene glycol, propylene glycol, glycerol, 1,4-butanediol, or 1,3-butanediol may be further added.

The organic solvent may be used in an amount of 200 parts by weight to 5,000 parts by weight, or 400 parts by weight to 3,000 parts by weight, with respect to 100 parts by weight of the base resin.

### <Base Resin>

The base resin may include a repeating unit containing an acid labile group represented by Formula 4 below:

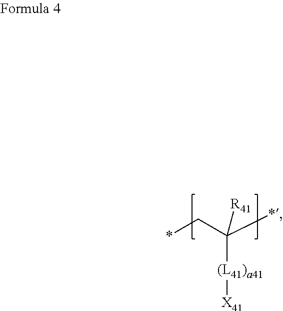

wherein, in Formula 4,
R₄₁ may be hydrogen, deuterium, a halogen, a C₁-C₂₀ linear or branched alkyl group, or a C₁-C₂₀ linear or branched halogenated alkyl group;
L₄₁ may be a single bond, O, C(═O), C(═O)O, OC(═O), C(═O)NH, NHC(═O), a substituted or unsubstituted C₁-C₃₀ alkylene group, a substituted or unsubstituted C₃-C₃₀ cycloalkylene group, a substituted or unsubstituted C₃-C₃₀ heterocycloalkylene group, a substituted or unsubstituted C₂-C₃₀ alkenylene group, a substituted or unsubstituted C₃-C₃₀ cycloalkenylene group, a substituted or unsubstituted C₃-C₃₀ heterocycloalkenylene group, a substituted or unsubstituted C₆-C₃₀ arylene group, or a substituted or unsubstituted C₁-C₃₀ heteroarylene group, or any combination thereof,
a41 may be an integer of 1 to 6,
X₄₁ may be an acid labile group,
* and *′ are each a binding site with a neighboring atom,
a substituent of the substituted C₁-C₃₀ alkylene group, the substituted C₃-C₃₀ cycloalkylene group, the substituted C₃-C₃₀ heterocycloalkylene group, the substituted C₂-C₃₀ alkenylene group, the substituted C₃-C₃₀ cycloalkenylene group, the substituted C₃-C₃₀ heterocycloalkenylene group, the substituted C₆-C₃₀ arylene group, or the substituted C₁-C₃₀ heteroarylene group may be:
deuterium, —F, —Cl, —Br, —I, —CD₃, —CD₂H, —CDH₂, —CF₃, —CF₂H, —CFH₂, a hydroxyl group, a carboxylic acid group or a salt thereof, a sulfonic acid group or a salt thereof, a phosphate group or a salt thereof, a C₁-C₂₀ alkyl group, a C₂-C₂₀ alkenyl group, a C₂-C₂₀ alkynyl group, or a C₁-C₂₀ alkoxy group;
a C₁-C₂₀ alkyl group, a C₂-C₂₀ alkenyl group, a C₂-C₂₀ alkynyl group, or a C₁-C₂₀ alkoxy group substituted with deuterium, —F, —Cl, —Br, —I, —CD₃, —CD₂H, —CDH₂, —CF₃, —CF₂H, —CFH₂, a hydroxyl group, a carboxylic acid group or a salt thereof, a sulfonic acid group or a salt thereof, a phosphate group or a salt thereof, a C₃-C₁₀ cycloalkyl group, a C₁-C₁₀ heterocycloalkyl group, a C₃-C₁₀ cycloalkenyl group, or a C₁-C₁₀ heterocycloalkenyl group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, a C₁-C₂₀ heteroarylthio group, a monovalent non-aromatic condensed polycyclic group, a monovalent non-aromatic heterocondensed polycyclic group, or any combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, a C₁-C₂₀ heteroarylthio group, a monovalent non-aromatic condensed polycyclic group, or a monovalent non-aromatic hetero condensed polycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₂₀ aryl group, a C₆-C₂₀ aryloxy group, a C₆-C₂₀ arylthio group, a C₁-C₂₀ heteroaryl group, a monovalent non-aromatic condensed polycyclic group, or a monovalent non-aromatic heterocondensed polycyclic group, substituted with deuterium, —F, —Cl, —Br, —I, —CD₃, —CD₂H, —CDH₂, —CF₃, —CF₂H, —CFH₂, a hydroxyl group, a carboxylic acid group or a salt thereof, a sulfonic acid group or a salt thereof, a phosphate group or a salt thereof, a C₁-C₂₀ alkyl group, a C₂-C₂₀ alkenyl group, a C₂-C₂₀ alkynyl group, a C₁-C₂₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₂₀ aryl group, a C₆-C₂₀ aryloxy group, a C₆-C₂₀ arylthio group, a C₁-C₆₀ heteroaryl group, a C₁-C₂₀ heteroaryloxy group, a C₁-C₂₀ heteroarylthio group, a monovalent non-aromatic condensed polycyclic group, a monovalent non-aromatic hetero condensed polycyclic group, or any combination thereof.

For example, in Formula 4, R₄₁ may be hydrogen, deuterium, a halogen, CH₃, CH₂F, CHF₂, or CF₃.

Examples of the “C₁-C₁₀ alkylene group” of L₄₁ in Formula 4 include a methylene group, an ethylene group, a propylene group, a butylene group, an isobutylene group, and the like.

Examples of the “C₃-C₁₀ cycloalkylene group” of L₄₁ in Formula 4 include a cyclopentylene group, a cyclohexylene group, an adamantylene group, an adamantylmethylene group, a norbornylene group, a norbornylmethylene group, a tricyclodecanylene group, a tetracyclododecanylene group, a tetracyclododecanylmethylene group, a dicyclohexylmethylene group, and the like.

A “C₁-C₁₀ heterocycloalkylene group” of L₄₁ in Formula 4 may be one in which some carbons of the “C₃-C₁₀ cycloalkylene group” are substituted with moieties containing heteroatoms, such as oxygen, sulfur, or nitrogen, and thus, the “C₁-C₁₀ heterocycloalkylene group” may contain an ether bond, an ester bond, a sulfonic acid ester bond, carbonate, a lactone ring, a sultone ring, or a carboxylic acid anhydride moiety.

a41 in Formula 4 refers to a number of repetitions of L₄₁, and when a41 is 2 or more, a plurality of L₄₁s may be identical to or different from each other.

In an embodiment, in Formula 4, X₄₁ may be represented by any one of the following Formulas 6-1 to 6-7:

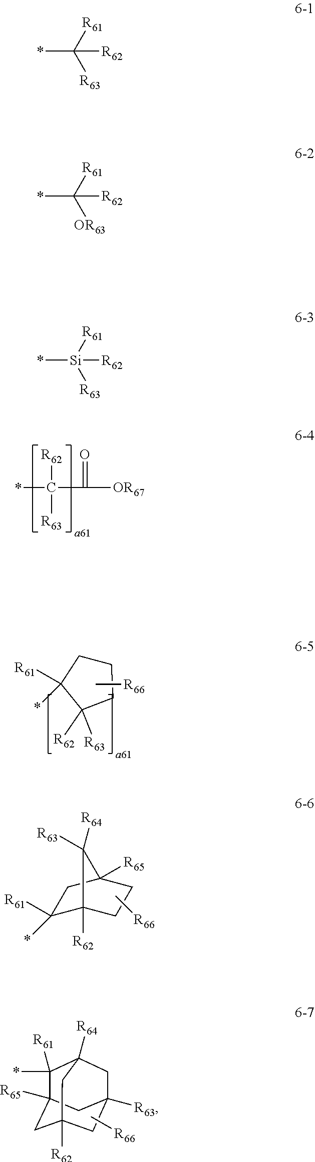

wherein, in Formulas 6-1 to 6-7,
a61 may be an integer of 0 to 6,
R₆₁ to R₆₆ may each independently be a linear, branched, or cyclic monovalent C₁-C₂₀ hydrocarbon group which may optionally contain hydrogen, deuterium, a halogen, a cyano group, a hydroxyl group, an amino group, a carboxylic acid group, or a heteroatom;
R₆₇ may be a linear, branched, or cyclic monovalent C₁-C₂₀ hydrocarbon group which may optionally contain a heteroatom,
two adjacent groups of R₆₁ to R₆₇ may be optionally bound to each other to form a ring, and
* may be a binding site with a neighboring atom.

In Formulas 6-4 and 6-5, when a61 is 0, (CR₆₂R₆₃)ₐ₆₁ is a single bond.

The “monovalent hydrocarbon group” of R₆₁ to R₆₇ in Formulas 6-1 to 6-7 may be understood by referring to the “monovalent hydrocarbon group” in the list of R₁₁ in Formula 1.

In an embodiment, the repeating unit represented by Formula 4 may be represented by any one of the following Formulas 4-1 and 4-2:

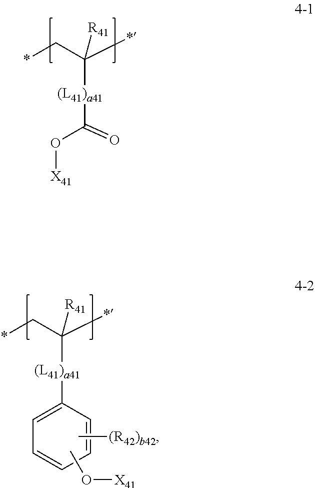

wherein, in Formulas 4-1 and 4-2,
each definition of L₄₁ and X₄₁ may be the same as defined for Formula 4,
a41 may be an integer of 1 to 4,
R₄₂ may be hydrogen, or a linear, branched, or cyclic monovalent C₁-C₂₀ hydrocarbon group which may optionally contain a heteroatom,
b42 may be an integer of 1 to 4, and
* and *′ are each a binding site with a neighboring atom.

The “monovalent hydrocarbon group” of R₄₂ in Formula 4-2 may be understood by referring to the “monovalent hydrocarbon group” in the list of R₁₁ in Formula 1.

The base resin containing the repeating unit represented by Formula 4 decomposes by an action of an acid to generate a carboxyl group, and therefore, is converted alkali-soluble.

The base resin may further include a repeating unit represented by Formula 5, apart from the repeating unit represented by Formula 4:

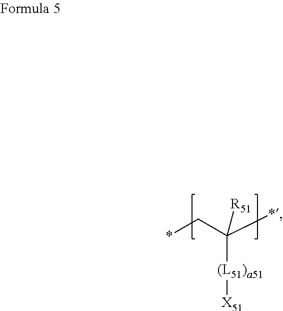

wherein, in Formula 5,
R₅₁ may be hydrogen, deuterium, a halogen, a linear or branched C₁-C₂₀ alkyl group, or a linear or branched C₁-C₂₀ halogenated alkyl group,

L₅₁ may be a single bond, O, C(═O), C(═O)O, OC(═O), C(═O)NH, NHC(═O), a substituted or unsubstituted C₁-C₃₀ alkylene group, a substituted or unsubstituted C₃-C₃₀ cycloalkylene group, a substituted or unsubstituted C₃-C₃₀ heterocycloalkylene group, a substituted or unsubstituted C₂-C₃₀ alkenylene group, a substituted or unsubstituted C₃-C₃₀ cycloalkenylene group, a substituted or unsubstituted C₃-C₃₀ heterocycloalkenylene group, a substituted or unsubstituted C₆-C₃₀ arylene group, or a substituted or unsubstituted C₁-C₃₀ heteroarylene group, or any combination thereof,
a51 may be an integer of 1 to 6,
X₅₁ may be a non-acid labile group, and
* and *′ are each a binding site with a neighboring atom, and
a substituent of the substituted C₁-C₃₀ alkylene group, the substituted C₃-C₃₀ cycloalkylene group, the substituted C₃-C₃₀ heterocycloalkylene group, the substituted C₂-C₃₀ alkenylene group, the substituted C₃-C₃₀ cycloalkenylene group, the substituted C₃-C₃₀ heterocycloalkenylene group, the substituted C₆-C₃₀ arylene group, or the substituted C₁-C₃₀ heteroarylene group may be the same as described for Formula 4.

For example, R₅₁ in Formula 5 may be understood by referring to the description of R₄₁ in Formula 4.

L₅₁ in Formula 5 may be understood by referring to the description of L₄₁ in Formula 4.

Also, a51 in Formula 5 refers to a number of repetitions of L₅₁, and when a51 is 2 or more, a plurality of L₅₁s may be identical to or different from each other.

In an embodiment, X₅₁ in Formula 5 may be hydrogen, or a linear, branched, or cyclic monovalent C₁-C₂₀ hydrocarbon group containing at least one polar moiety selected from a hydroxyl group, a halogen, a cyano group, a carbonyl group, a carboxyl group, *13 O—*′, *—C(═O)O—*′, —OC(═O)—*′, *—S(═O)O—*′, —OS(═O)—*′, a lactone ring, a sultone ring, and a carboxylic acid anhydride moiety. Here, “monovalent hydrocarbon group” may be understood by referring to “monovalent hydrocarbon group” in the list of R₁₁ in Formula 1, and the monovalent hydrocabon groups must contain at least one polar moiety selected from a hydroxyl group, a halogen, a cyano group, a carbonyl group, a carboxyl group, *—O—*′, *—C(═O)O—*′, —OC(═O)—*′, *—S(═O)O—*′, —OS(═O)—*′, a lactone ring, a sultone ring, and a carboxylic acid anhydride moiety.

In an embodiment, the repeating unit represented by Formula 5 may be represented by any one of the following Formulas 5-1 and 5-2:

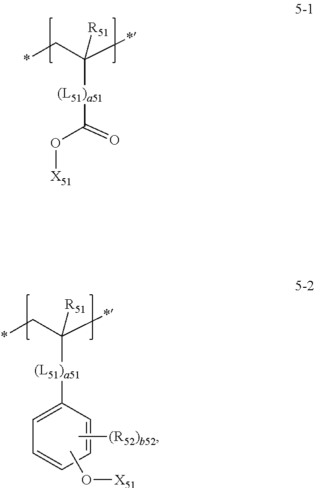

wherein in Formulas 5-1 and 5-2,
each definition of L₅₁ and X₅₁ may be the same as defined for Formula 5, a51 may be an integer of 1 to 4, R₅₂ may be hydrogen, a hydroxyl group, or a linear, branched, or cyclic monovalent C₁-C₂₀ hydrocarbon group which may optionally contain a heteroatom, b52 may be an integer of 1 to 4, and
* and *′ are each a binding site with a neighboring atom.

The “monovalent hydrocarbon group” of R₅₂ in Formula 5-2 may be understood by referring to the “monovalent hydrocarbon group” in the list of R₁₁ in Formula 1.

For example, in an ArF lithography process, X₅₁ may contain a lactone ring as a polar moiety, and in KrF, EB and EUV lithography processes, X₅₁ may be phenol.

In an embodiment, the base resin may further include a moiety including an anion and/or a cation. For example, the base resin may further include a moiety in which a photoacid generator and/or a quencher are induced to bind to a side chain.

The base resin may have a weight average molecular weight (Mw) of about 1,000 to about 500,000, or about 3,000 to about 100,000, as measured by gel permeation chromatography, using a tetrahydrofuran solvent, and polystyrene as a standard material.

A polydispersity index (PDI: Mw/Mn) of the base resin may be about 1.0 to about 3.0, or about 1.0 to about 2.0. When the above-described range is satisfied, a possibility of foreign matter remaining on the pattern may be reduced, or deterioration of the pattern profile may be minimized. Accordingly, the photoresist composition may become more suitable for forming fine patterns.

The base resin may be prepared by any suitable method, for example, by dissolving unsaturated bond-containing monomers in an organic solvent, and by thermal polymerizing in a presence of a radical initiator.

In the base resin, mole fractions (mol %) of each repeating unit derived from each monomer are as follows, but are not limited thereto:

i) about 1 mol % to about 60 mol %, about 5 mol % to about 50 mol %, or about 10 mol % to about 50 mol % of the repeating unit represented by Formula 4 is included;

ii) about 40 mol % to about 99 mol %, about 50 mol % to about 95 mol %, or about 50 mol % to about 90 mol % of the repeating unit represented by Formula 5 is included.

The base resin may be a single polymer or may include a mixture of two or more polymers different in composition, weight average molecular weight, and/or polydispersity index.

### <Photoacid Generator>

When the organic salt is decomposed by exposure and generates an acid, the organic salt may act as a photoacid generator, and thus, the photoresist composition may not include a separate photoacid generator.

The photoacid generator may be any compound capable of generating acid when exposed to high-energy rays such as UV, DUV, EB, EUV, X-rays, α-rays, and γ-rays.

The photoacid generator may be included in an amount of 0 parts by weight to about 80 parts by weight, about 0.1 parts by weight to about 80 parts by weight, or about 0.1 parts by weight to about 60 parts by weight, with respect to 100 parts by weight of the base resin. When the above range is satisfied, appropriate resolution may be achieved, and problems related to foreign particles after development or during stripping may be reduced.

One type of the photoacid generator may be used, or two or more different types may be used in combination.

### <Quencher>

The quencher may be a salt that generates an acid having a weaker acidity than the acid generated from the photoacid generator.

The quencher may include an ammonium salt, a sulfonium salt, an iodonium salt, and combinations thereof.

In an embodiment, the quencher may be represented by Formula 8 below:

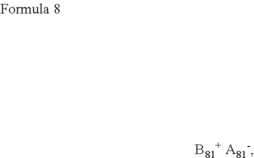

wherein in Formula 8,
B₈₁⁺ is represented by any one of the following Formulas 8A to 8C,
A₈₁⁻ is represented by any one of the following Formulas 8D to 8F,
B₈₁⁺ and A₈₁⁻ may be optionally linked via a carbon-carbon covalent bond;

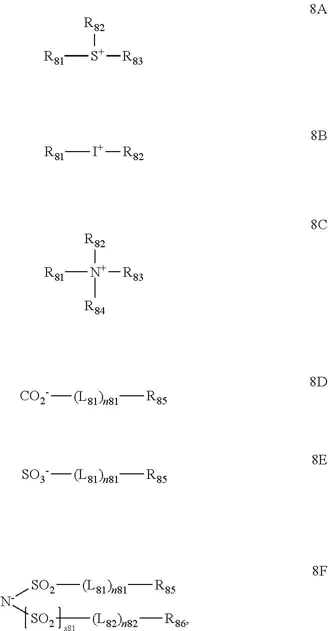

wherein in Formulas 8A to 8F,
R₈₁ to R₈₄ are each independently a linear, branched, or cyclic monovalent C₁-C₂₀ hydrocarbon group,
two adjacent groups of R₈₁ to R₈₄ may be optionally bound to each other to form a ring,

L₈₁ and L₈₂ are each independently a single bond, or CRR′,
R and R′ may each independently be hydrogen, deuterium, a halogen, a hydroxyl group, a C₁-C₂₀ alkyl group, a C₁-C₂₀ halogenated alkyl group, a C₁-C₂₀ alkoxy group, a C₃-C₂₀ cycloalkyl group, or a C₃-C₂₀ cycloalkoxy group,
n81 and n82 are each independently 1, 2, or 3,
n81 is 0 or 1, and
R₈₅ and R₈₆ may be a linear, branched, or cyclic monovalent C₁-C₃₀ hydrocarbon group which may optionally contain hydrogen, a halogen, or a heteroatom.

The quencher may be included in an amount of about 0.01 parts by weight to about 40 parts by weight, about 0.05 parts by weight to about 40 parts by weight, or about 0.1 parts by weight to about 20 parts by weight, with respect to 100 parts by weight of the base resin. When the above range is satisfied, appropriate resolution may be achieved, and problems related to foreign particles after development or during stripping may be reduced.

For the quencher, one type may be used, or two or more different types may be used in combination.

### <Optional Ingredients>

The photoresist composition may further include a surfactant, a crosslinker, a leveling agent, a colorant, or any combination thereof, as needed.

The photoresist composition may further include a surfactant to improve coatability, developability, etc. Specific examples of the surfactant include, for example, nonionic surfactants such as polyoxyethylene lauryl ether, polyoxyethylene stearyl ether, polyoxyethylene oleyl ether, polyoxyethylene n-octylphenyl ether, polyoxyethylene n-nonylphenyl ether, polyethyleneglycol dilaurate, polyethyleneglycol distearate, etc. As the surfactant, a commercially available product or a synthetic product may be used. Examples of commercially available surfactants include, for example, KP341 (manufactured by Shin-Etsu Chemical Co., Ltd.), Polyflow No. 75, Polyflow No. 95 (above, manufactured by Kyoeisha Chemical Co., Ltd.), F-top EF301, F-top EF303, F-top EF352 (above, manufactured by Mitsubishi Material Electronic Chemicals Co., Ltd.), MEGAFACE (registered trademark) F171, MEGAFACE F173, R40, R41, R43 (above, manufactured by DIC Corporation), Fluorad (registered trademark) FC430, Fluorad FC431 (above, manufactured by 3M), AsahiGuard AG710 (manufactured by AGC Corporation), Surflon (registered trademark) S-382, Surflon SC-101, Surflon SC-102, Surflon SC-103, Surflon SC-104, Surflon SC-105, Surflon SC-106 (above, manufactured by AGC Seimi Chemical Co., Ltd.), etc.

The surfactant may be included in an amount of about 0 parts by weight to about 20 parts by weight, with respect to 100 parts by weight of the base resin. One type of the surfactant may be used, or two or more different types may be used in combination.

A method of preparing the photoresist composition is not particularly limited, and for example, a method of mixing an organic salt, a base resin, a photoacid generator, and optional components as needed in an organic solvent may be used. A mixting temperature or mixing time is not particularly limited. Filtration may be performed after mixing, as needed.

### Pattern Formation Method

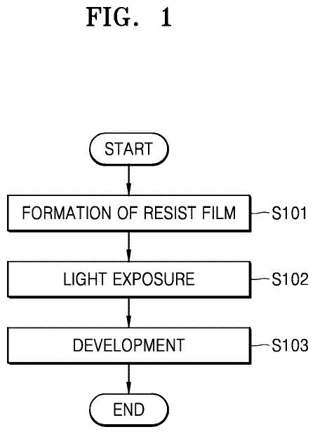

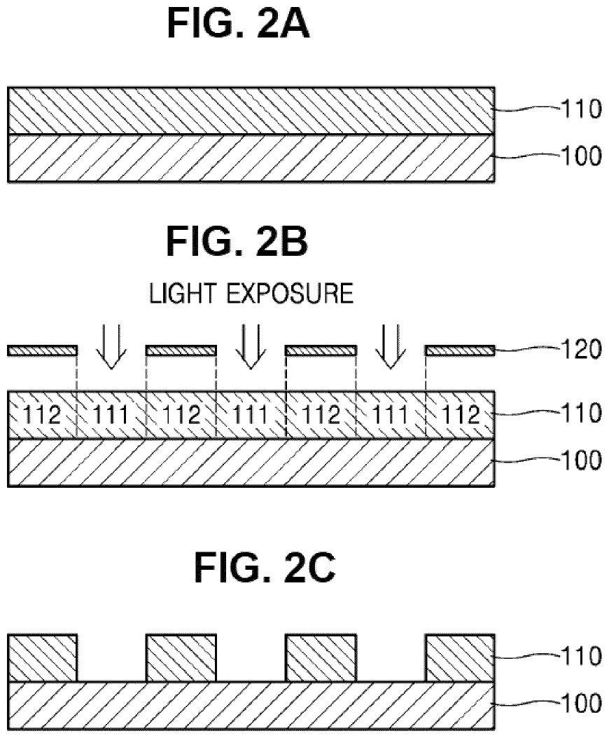

Hereinafter, a method of forming a pattern according to embodiments will be described in more detail with reference to FIGS. 1 and 2. FIG. 1 is a flow chart illustrating a method of forming a pattern according to embodiments, and FIGS. 2A to 2C are side cross-sectional views illustrating the pattern formation method according to embodiments. Hereinafter, a method of forming a pattern using a positive photoresist composition will be described as an example, but the method is not limited thereto.

Referring to FIG. 1, the pattern formation method includes: forming a resist film by applying a photoresist composition (S101); exposing at least a portion of the photoresist film with high-energy rays (S102); and developing the exposed photoresist film by using a developing solution (S103). The above processes may be omitted when necessary, or may be performed in different order.

First, referring to FIG. 2A, a substrate 100 is prepared. For the substrate 100, for example, a semiconductor substrate such as a silicon substrate or a germanium substrate, glass, quartz, ceramic, copper, etc. may be used. In one or more embodiments, the substrate 100 may include a III-V compound such as GaP, GaAs, GaSb, etc.

Referring to FIG. 1, S101, the photoresist film 110 may be formed by applying a photoresist composition to a desired thickness on the substrate 100 by a coating method. When necessary, heating may be performed to remove an organic solvent remaining in the photoresist layer 110. As the coating method, spin coating, dipping, roller coating, or other general coating methods may be used. Among these, spin coating may be particularly used, and the photoresist film 110 having a desired thickness may be formed by adjusting viscosity, concentration, and/or spin speed of the photoresist composition. In some embodiments, the photoresist film 110 may have a thickness of about 10 nm to about 300 nm. In some embodiments, the photoresist film 110 may have a thickness of about 15 nm to about 200 nm.

A lower limit of a prebaking temperature may be about 60° C. or higher, or about 80° C. or higher. In addition, an upper limit of the prebaking temperature may be 150° C. or less, or 140° C.or less. A lower limit of prebaking time may be about 5 seconds or higher, or about 10 seconds or higher. An upper limit of the prebaking time may be about 600 seconds or less, or about 300 seconds or less.

Prior to applying the photoresist composition on the substrate 100, an etching target film (not shown) may be further formed on the substrate 100. The etching target film may refer to a layer on which an image is transferred from a photoresist pattern and converted to a desired and/or alternatively predetermined pattern. In an embodiment, the etching target film may be formed to include an insulating material such as silicon oxide, silicon nitride, or silicon oxynitride. In one or more embodiments, the etching target film may be formed to include a conductive material such as a metal, a metal nitride, a metal silicide, or a metal silicide nitride film. In one or more embodiments, the etching target film may be formed to include a semiconductor material such as polysilicon.

In an embodiment, an anti-reflection film may be further formed on the substrate 100 to maximize efficiency of the photoresist. The anti-reflection film may be an organic or inorganic anti-reflection film.

In an embodiment, a protective film may be further provided on the photoresist film 100 to reduce effects of alkaline impurities or the like included in the process. In addition, when immersion exposure is performed, for example, a protective film for immersion may be provided on the photoresist film 100 to avoid direct contact between the immersion medium and the photoresist film 100.

Next, referring to FIG. 1 (S102) and FIG. 2B, at least a portion of the photoresist film 110 may be exposed to high-energy rays. For example, high-energy rays passing through the mask 120 may be irradiated to at least a portion of the photoresist film 110. For this reason, the photoresist film 110 may have an exposed portion 111 and a non-exposed portion 112.

In some cases, this exposure is performed by irradiating high-energy rays through a mask having a desired and/or alternatively predetermined pattern, using a liquid such as water as a medium. Examples of the high-energy rays include electromagnetic waves such as ultraviolet rays, far-ultraviolet rays, extra-ultraviolet rays (EUV, wavelength 13.5 nm), X-rays, and γ-rays; charged corpuscular beams such as electron beams (EB), α rays, etc. Irradiation of these high-energy rays may be collectively referred to as “exposure”.

As a light source for the exposure, various devices such as a device emitting laser light in the ultraviolet region such as a KrF excimer laser (wavelength 248 nm), an ArF excimer laser (wavelength 193 nm), and F₂ excimer laser (wavelength 157 nm); a device that converts wavelengths of laser light from a solid-state laser light source (YAG or semiconductor laser, etc.) to emit harmonic laser light in the far-ultraviolet region or vacuum ultraviolet region; or a device that irradiates electron beams or extreme ultraviolet (EUV) rays may be used. Exposure is usually performed through a mask corresponding to a desired pattern, but when the exposure light source is electron beams, exposure may be performed by direct drawing without using a mask.

An integrated dose of high-energy rays, for example, when extreme ultraviolet rays are used as the high-energy rays, may be about 2,000 mJ/cm² or less, or about 500 mJ/cm² or less. In addition, when electron beams are used as the high-energy rays, an integrated dose may be about 5,000 μC/cm² or less, or about 1,000 μC/cm² or less.

In addition, post exposure bake (PEB) may be performed after an exposure. A lower limit of a temperature at which PEB is performed may be about 50° C.or higher, or about 80° C. or higher. An upper limit of the temperature at which PEB is performed may be about 180° C. or lower, or about 130° C. or lower. A lower limit of time of performing PEB may be about 5 seconds or more, or about 10 seconds or more. An upper limit of time of performing PEB may be about 600 seconds or less, or about 300 seconds or less.

Next, referring to FIG. 1 (S103) and FIG. 2C, the exposed photoresist film 110 may be developed by using a developing solution. The exposed portion 111 may be washed away by the developing solution, and the non-exposed portion 112 remains unwashed away by the developing solution.

Examples of the developing solution include an alkaline developing solution, a developing solution containing an organic solvent (hereinafter, also referred to as “organic developing solution”), etc. Examples of the developing method include the dipping method, the puddle method, the spray method, the dynamic dosing method, etc. A developing temperature may be, for example, about 5° C. or more to about 60° C. or less, and the developing time may be, for example, about 5 seconds or more to about 300 seconds or less.

The alkaline developing solutions include, for example, alkaline aqueous solutions in which at least one type of alkaline compounds is dissolved, the alkaline compounds including: sodium hydroxide, potassium hydroxide, sodium carbonate, sodium silicate, sodium metasilicate, ammonia water, ethylamine, n-propylamine, diethylamine, di-n-propylamine, triethylamine, methyldiethyl amine, ethyldimethylamine, triethanolamine, tetramethyl ammonium hydroxide (TMAH), pyrrole, piperidine, choline, 1,8-diazabicyclo[5.4.0]-7-undecene (DBU), 1,5-diazabicyclo[4.3.0]-5-nonene (DBN), etc. The alkaline developing solution may further include a surfactant.

A lower limit of a content of the alkaline compounds in the alkaline developing solution may be about 0.1 wt % or more, about 0.5 wt % or more, or about 1 wt % or more. In addition, an upper limit of a content of the alkaline compounds in the alkaline developing solution may be about 20 wt % or less, about 10 wt % or less, or about 5 wt % or less.

After development, the photoresist pattern may be washed with ultrapure water, and then water remaining on the substrate and the pattern may be removed.

As an organic solvent included in the organic developing solution, for example, the same organic solvent as described in the <Organic solvent> section of [Photoresist composition] may be used.

A lower limit of a content of the organic solvent in the organic developing solution may be about 80 wt % or more, about 90 wt % or more, about 95 wt % or more, or about 99 wt % or more.

The organic developing solution may further include a surfactant. In addition, a trace amount of water may be included in the organic developing solution. In addition, during development, development may also be stopped by substituting the organic developing solution with a solvent of a different kind.

After the development, the photoresist pattern may be further cleaned. Ultrapure water, a rinse solution, etc. may be used as a cleaning liquid. The rinsing solution is not particularly limited as long as it does not dissolve the photoresist pattern, and a solution including a general organic solvent may be used. For example, the rinsing solution may be an alcohol-based solvent or an ester-based solvent. After cleaning, the rinsing solution remaining on the substrate and pattern may be removed. In addition, when ultrapure water is used, water remaining on the substrate and the pattern may be removed.

In addition, one type of a developing solution may be used alone, or two or more types of developing solutions may be used in combination.

After the photoresist pattern is formed as described above, a pattern wiring substrate may be obtained by etching. The etching may be performed by known methods such as dry etching using a plasma gas and wet etching using an alkaline solution, cupric chloride solution, ferric chloride solution, or the like.

After forming the resist pattern, plating may be performed. The plating method is not particularly limited, and examples thereof include copper plating, solder plating, nickel plating, and gold plating.

The photoresist pattern remaining after etching may be stripped with an organic solvent. Examples of such an organic solvent include, but are not particularly limited to, propylene glycol monomethyl ether acetate (PGMEA), propylene glycol monomethyl ether (PGME), ethyl lactate (EL), and the like. The stripping method is not particularly limited, but includes, for example, an immersion method, a spray method, etc. Further, the wiring substrate on which the photoresist pattern is formed may be a multilayer wiring substrate, or may have small-diameter through-holes.

In an embodiment, the wiring substrate may be formed by a method of depositing a metal in a vacuum after forming a photoresist pattern and then melting the photoresist pattern with a solution, that is, a lift-off method.

The present disclosure will be described in more detail using the following examples and comparative examples, but the technical scope of the present disclosure is not limited only to the following examples.

### EMBODIMENT

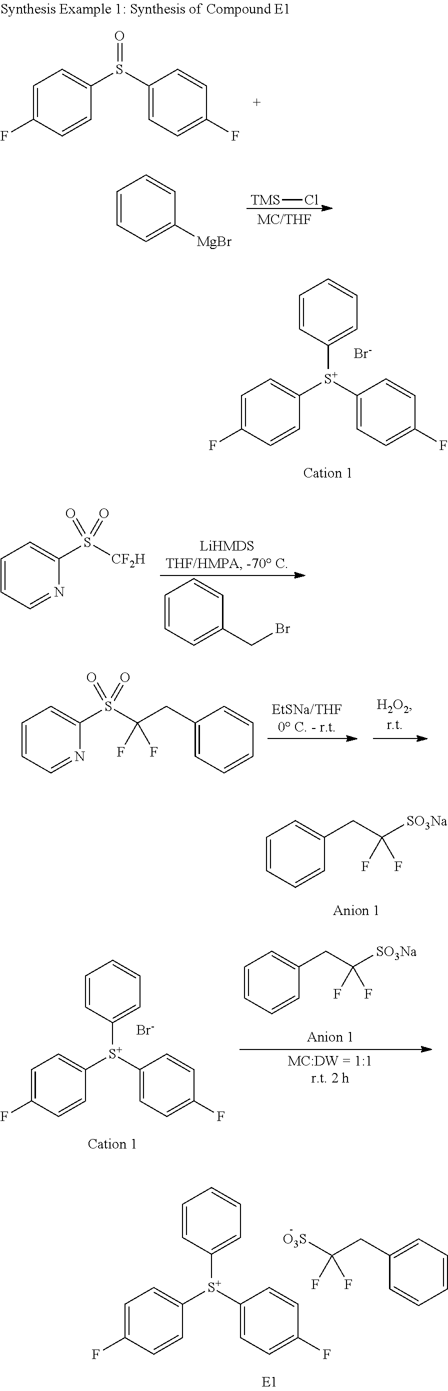

### Synthesis of Cation 1

2.38 g (10.0 mmol) of 4,4′-sulfinylbisfluorobenzene was added into 20 ml of dichloromethane (DCM) and maintained at 0° C. After slowly adding 3.25 g (30.0 mmole) of chlorotrimethylsilane dropwise, the mixture was stirred at room temperature for 30 minutes. 10 mL (29.0 mmole) of 2.9 M phenylmagnesium bromide was slowly added dropwise and stirred at room temperature for 1 hour. After completing the reaction by adding 2.5 g of water, the organic layer was washed with an aqueous hydrogen chloride solution, dried over anhydrous Na₂SO₄, and concentration dried. Cation1 was obtained after purification by recrystallization (i-PrOH-hexane). The resulting compound was confirmed by NMR and LC-MS.

¹H NMR (500 MHZ, CD₂Cl₂): δ 7.41-7.44 (m, 4H), 7.72-7.73 (m, 2H), 7.80-7.81 (m, 3H), 7.98-8.00 (m, 4H), LC-MS m/z=299.0605 (cation).

### Synthesis of Anion 1

Benzyl bromide (10.3 g, 60.2 mmol), tetrahydrofuran (260 mL), 2-difluoromethyl sulfonylpyridine (9 g, 46.6 mmol), and hexamethyl phosphoamide (26 mL) were added to a flask under nitrogen atmosphere, and the reaction mixture was cooled to −70° C. Lithium hexamethyldisilazide tetrahydrofuran solution (LiHMDS in THF) (70 ml, 70 mmol) was added dropwise to the mixture. After stirring for 1 hour, the reaction was quenched with saturated NH₄Cl. After adding water, the mixture was extracted with ethyl acetate, and an organic layer was obtained and dried over MgSO₄. After removing the solvent under reduced pressure, the product was purified by flash column chromatography (hexane/ethyl acetate, 2:1) to obtain a product 2-(1,1-difluoro-2-methylethyl)sulfonylpyridine (yield 27%).

¹H NMR (400 MHZ, CDCl₃): δ 3.73 (t, 2H), 7.26-7.38 (m, 5H), 7.64-7.69 (m, 1H), 8.02-8.06 (m, 1H), 8.17-8.20 (m, 1H), 8.86-8.88 (m, 1H)

Under nitrogen atmosphere, 2-(1,1-difluoro-2-methylethyl)sulfonyl pyridine (4 g) and 40 ml of tetrahydrofuran were added to the flask and maintained at 0° C. After adding sodium ethenethiolate (2.35 g) at 0° C.for 5 minutes, the mixture was stirred at 0° C. for about 2 hours and then stirred at room temperature for 10 hours. After the reaction, the solvent was removed, and the residue was extracted with water and ether to remove 2-(ethylthio)pyridine and ethanethiol. Then, a hydrogen peroxide solution (30 wt %, 4 mL) was added to the water layer and stirred at room temperature for 12 hours. After removing the solvent under reduced pressure, the product was dissolved in ether and filtered to remove insoluble salts. The filtrate was concentrated to obtain Anion 1.

¹H NMR (400 MHZ, CD₃OD): δ 3.44-3.57 (m, 2H), 7.35-7.41 (m, 5H), LC-MS m/z=221.0205 (anion).

### Synthesis of Compound E1

Cation 1 (0.5 g, 1.32 mmol) and Anion 1 (0.34 g, 1.38 mmol) were mixed with 10 mL of dichloromethane (DCM) and 10 mL of DW, followed by stirring for 2 hours. Thereafter, the organic layer was separated, dried over anhydrous Na₂SO₄, filtered, and the filtrate obtained therefrom was removed, and the obtained residue was separated and purified by silica gel column chromatography to obtain Example 1 (0.2 g, 29%). The resulting compound was confirmed by NMR.

¹H NMR (500 MHZ, CD₂Cl₂): δ 3.44-3.51 (t, 2H), 7.26 (m, 5H), 7.38-7.41 (m, 4H), 7.67-7.69 (m, 4H), 7.78-7.82 (m, 5H)

### Synthesis Example 2

### Synthesis of Compound E2

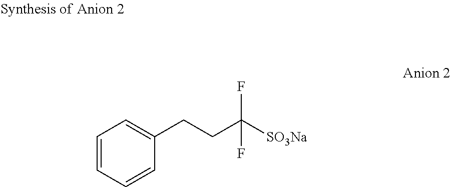

Anion 2 was synthesized by using the same method as in the synthesis of Anion 1 in Synthesis Example 1, except that (2-bromoethyl)benzene was used instead of benzyl bromide. The resulting compound was confirmed by NMR and LC-MS.

¹H NMR (400 MHZ, CD₃OD): δ 2.39-2.57 (m, 2H), 2.82-2.92 (m, 2H), 7.22-7.34 (m, 5H), LC-MS m/z=235.0602 (anion).

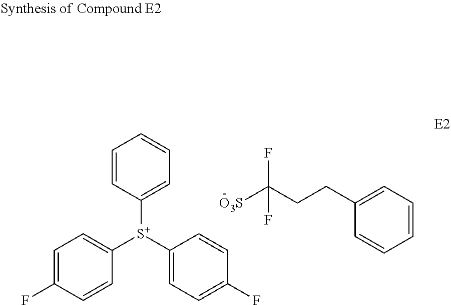

Compound E2 was synthesized by using the same method as in the synthesis of Compound E1, except that Anion 2 was used instead of Anion 1. The resulting compound was confirmed by NMR.

¹H NMR (500 MHZ, CD₂Cl₂): δ 2.44-2.51 (m, 2H), 2.85-2.89 (m, 2H), 7.19 (m, 3H), 7.24 (m, 2H), 7.42-7.44 (m, 4H), 7.69 (m, 4H), 7.71 (m, 1H), 7.81-7.83 (m, 4H)

### Synthesis Example 3

### Synthesis of Compound E3

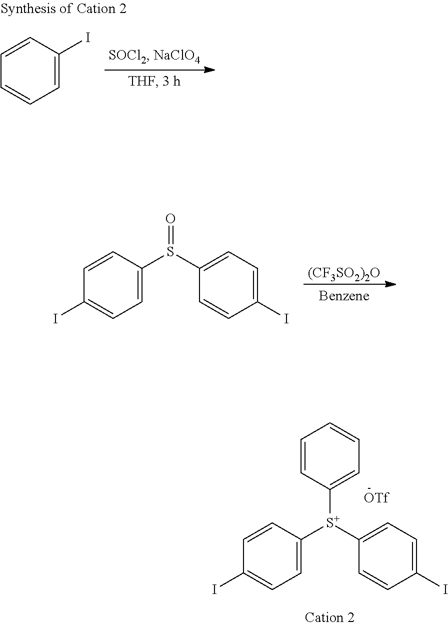

4-iodobenzene (2.246 g, 11.01 mmol), thionyl chloride (0.655 g, 5.51 mmol), and sodium perchlorate (0.117 g, 1.10 mmol) were mixed in 12 mL of tetrahydrofuran, and stirred for 3 hours. Thereafter, the reaction solvent was removed by distillation under reduced pressure, and an organic layer obtained by extraction with 30 ml of water and 30 mL of dichloromethane was dried over Na₂SO₄ and filtered. The filtrate obtained therefrom was subjected to reduced pressure, and the residue obtained thereby was separated and purified by column chromatography to obtain a compound 4,4′-sulfinylbis(iodobenzene). The resulting compound was confirmed by NMR.

¹H NMR (300 MHz, CDCl₃): δ 7.05 (d, 4H), 7.42 (d, 4H)

After dissolving the compound 4,4′-sulfinylbis(iodobenzene) (3.73 g, 8.20 mmol) in 15 mL of benzene, trifluoromethane sulfonic anhydride (2.778 g, 9.85 mmol) was added dropwise at 0° C. and stirred at room temperature for 1 hour. Thereafter, the organic layer obtained by extraction with 20 ml of water and 50 mL of ethyl acetate was washed with a saturated NaHCO₃ aqueous solution, dried over MgSO₄, and filtered. The filtrate obtained therefrom was subjected to reduced pressure, and the residue obtained thereby was separated and purified by column chromatography to obtain a compound Cation 2 (4.92 g, 90%). The resulting compound was confirmed by NMR and LC-MS.

¹H NMR (500 MHZ, CD₂Cl₂): δ 7.37-7.40 (m, 4H), 7.65-7.67 (m, 2H), 7.73 (t, 2H), 7.81 (t, 1H), 8.06-8.08 (m, 4H), LC-MS m/z=515.8326 (cation).

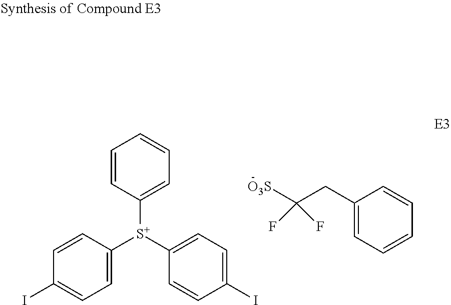

Compound E3 was synthesized by using the same method as in the synthesis of Compound E1, except that Cation 2 was used instead of Cation 1. The resulting compound was confirmed by NMR.

¹H NMR (500 MHZ, CD₂Cl₂): δ 3.45-3.53 (t, 2H), 7.29 (m, 5H), 7.42-7.44 (m, 4H), 7.7 (m, 4H), 7.8 (m, 1H), 8.04-8.05 (m, 4H)

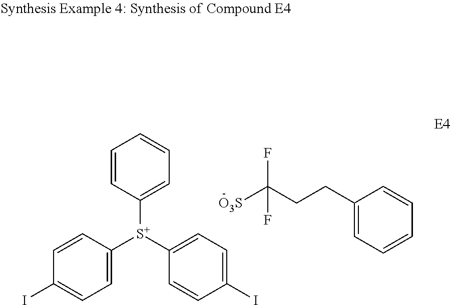

Compound E4 was synthesized by using the same method as in the synthesis of Compound E3, except that Anion 2 was used instead of Anion 1. The resulting compound was confirmed by NMR.

¹H NMR (500 MHZ, CD₂Cl₂): δ 2.4-2.5 (m, 2H), 2.85-2.89 (m, 2H), 7.19 (m, 3H), 7.26 (m, 2H), 7.45 (m, 4H), 7.7 (m, 4H), 7.8 (m, 1H), 8.03 (m, 4H)

### Synthesis Example 5

### Synthesis of Compound E5

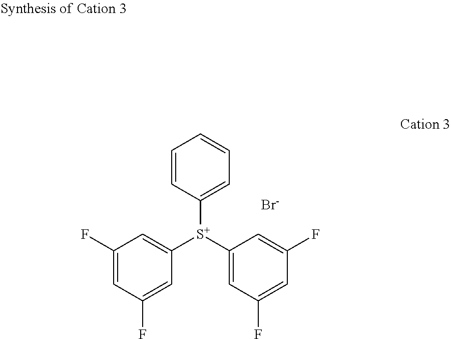 [SMILES: Fc1cc(F)cc([S+](c2ccccc2)c2cc(F)cc(F)c2)c1 ; [Br-]]

Cation 3 was synthesized by using the same method as in the synthesis of Cation 2, except that 1-bromo-3,5-difluorobenzene was used instead of 1-iodobenzene. The resulting compound was confirmed by NMR and LC-MS.

¹H NMR (500 MHZ, CD₂Cl₂): δ 7.24 (m, 2H), 7.63 (m, 4H), 7.74-7.76 (m, 2H), 7.83 (m, 1H), 7.94-7.96 (m, 2H), LC-MS m/z=336.0238 (cation).

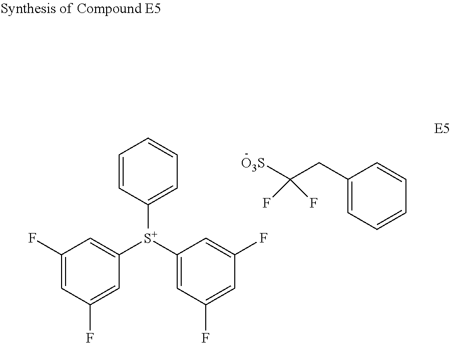

Compound E5 was synthesized by using the same method as in the synthesis of Compound E1, except that Cation 3 was used instead of Cation 1. The resulting compound was confirmed by NMR.

¹H NMR (500 MHZ, CD₂Cl₂): δ 3.46-3.55 (t, 2H), 7.29 (m, 7H), 7.39 (m, 4H), 7.77 (m, 4H), 7.87 (m, 1H).

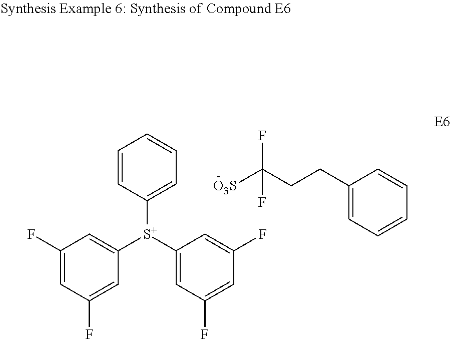

Compound E6 was synthesized by using the same method as in the synthesis of Compound E5, except that Anion 2 was used instead of Anion 1. The resulting compound was confirmed by NMR.

¹H NMR (500 MHZ, CD₂Cl₂): δ 2.39-2.49 (m, 2H), 2.85-2.88 (m, 2H), 7.18 (−7.19m, 3H), 7.26-7.27 (m, 4H), 7.42 (m, 4H), 7.76-7.78 (m, 2H), 7.84 (m, 3H)

### Evaluation Example 1

### Evaluation of Acid Generating Effect

10 wt % of poly(4-vinylphenol) (Mw: about 11k) and 6.5 wt % of coumarin 6 were dissolved in cyclohexanone at this ratio, and compounds of Tables 1 to 3 were added in the same mole number as coumarin to form a polymer solution. This solution was spin coated on 1 inch-quartz to a thickness of 400 nm and dried at 130° C. for 2 minutes to form a thin film. After exposing the thin film to DUV (248 nm) at 10 mJ/cm² to 100 mJ/cm², absorbance was measured.

Assuming that the absorbance of coumarin increases at a wavelength of 535 nm due to acid generation, and that coumarin, an acid indicator, was 100% converted after exposure at 100 mJ, intensity after exposure to 20 mJ was normalized with the intensity after exposure at 100 mJ, to compare degrees of acid generation at each thin film.

TABLE 1

| | | Degree of acid |
|---|---|---|
| | | generation (%) |
| | Compound structure | @ DUV 20 mJ |
| Example 1 | Compound E1 | 67.4 |
| Example 2 | Compound E2 | 64.3 |
| Comparative Example 1 | 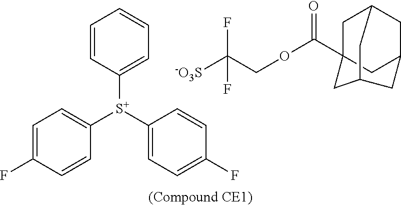 | 56.6 |
| Comparative Example 2 | 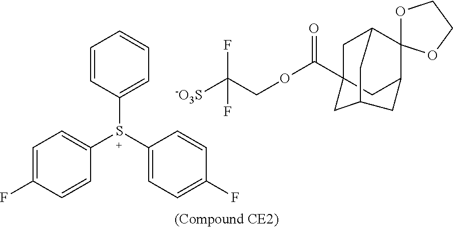 | 61.7 |
| Comparative Example 3 | 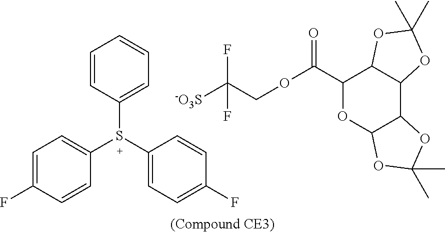 | 58.6 |
| Comparative Example 4 | 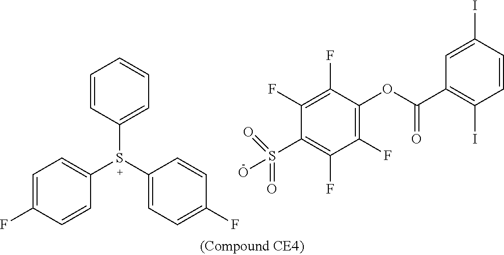 | 57.0 |

TABLE 2

| | | Degree of acid |
|---|---|---|
| | | generation (%) |
| | Compound structure | @ DUV 20 mJ |
| Example 3 | Compound E3 | 60.2 |
| Example 4 | Compound E4 | 56.5 |
| Comparative Example 5 | 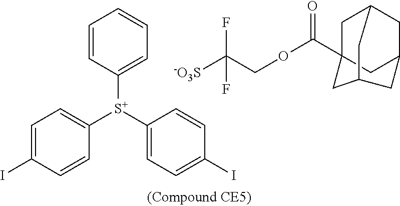 | 55.8 |
| Comparative Example 6 | 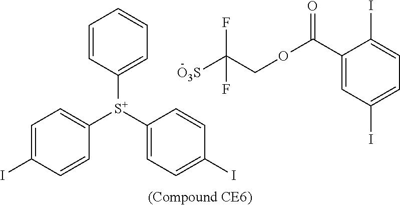 | 55.5 |
| Comparative Example 7 | 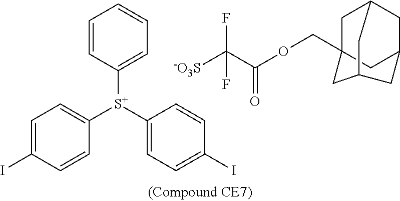 | 56.2 |
| Comparative Example 8 | 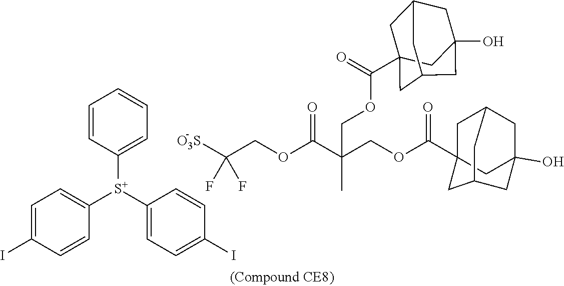 | 56.0 |

TABLE 3

| | | Degree of acid |
|---|---|---|
| | | generation (%) |
| | Compound structure | @ DUV 20 mJ |
| Example 5 | Compound E5 | 69.0 |
| Example 6 | Compound E6 | 68.5 |
| Comparative Example 9 | 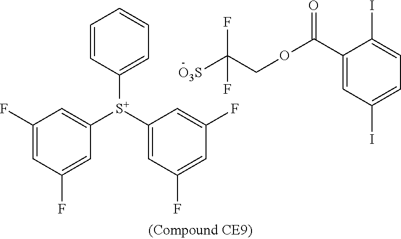 | 63.7 |
| Comparative Example 10 | 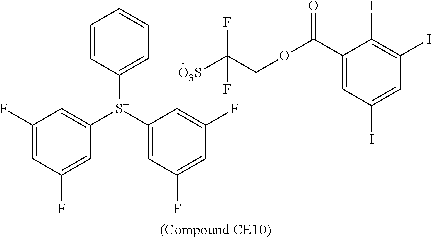 | 65.4 |
| Comparative Example 11 | 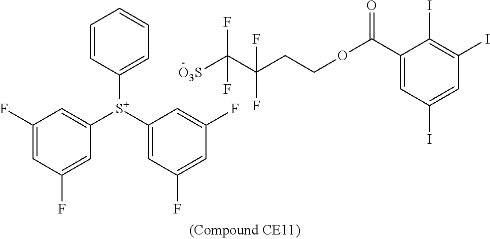 | 63.6 |
| Comparative Example 12 | 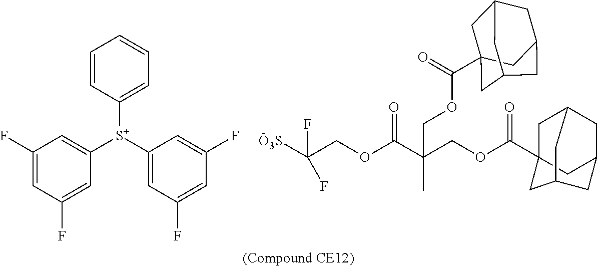 | 65.0 |

Referring to Tables 1 to 3, it may be confirmed that Examples 1 and 2 exhibit superior acid generating effects compared to Comparative Examples 1 to 4, Examples 3 and 4 exhibit superior acid generating effects compared to Comparative Examples 5 to 8, and Examples 5 and 6 exhibit excellent acid generating effects compared to Comparative Examples 9 to 12.

### Evaluation Example 2

### Ohnishi Parameter

Ohnishi parameters of compounds E1 to E6 and compounds CE1 to CE2 were calculated by using Equation 1 below, and the results are shown in Tables 4 to 6 below. The smaller the value of the Ohnishi parameter, the better the etching resistance.

$$OP = \frac{{N}_{tot}}{{N}_{C} - {N}_{O}} \qquad Equation 1$$

(In Equation 1, OP refers to an Ohnishi parameter, Nₜₒₜ refers to a total number of atoms in a compound, N_{C} refers to a number of carbon atoms in the compound, and N_{O} refers to a number of oxygen atoms in the compound)

TABLE 4

| Compound | Formula | Ohnishi parameter |
|---|---|---|
| Compound E1 | C₂₆H₂₀F₄O₃S₂ | 2.391 |
| Compound E2 | C₂₇H₂₂F₄O₃S₂ | 2.417 |
| Compound CE1 | C₃₁H₃₀F₄O₅S₂ | 2.769 |
| Compound CE2 | C₃₃H₃₂F₄O₇S₂ | 3.000 |
| Compound CE3 | C₃₂H₃₂F₄O₁₀S₂ | 3.636 |
| Compound CE4 | C₃₁H₁₆F₆I₂O₅S₂ | 2.385 |

TABLE 5

| Compound | Formula | Ohnishi parameter |
|---|---|---|
| Compound E3 | C₂₆H₂₀F₂I₂O₃S₂ | 2.391 |
| Compound E4 | C₂₇H₂₂F₂I₂O₃S₂ | 2.417 |
| Compound CE5 | C₃₁H₃₀F₂I₂O₅S₂ | 2.769 |
| Compound CE6 | C₂₇H₁₈F₂I₄O₅S₂ | 2.636 |
| Compound CE7 | C₃₁H₃₀F₂I₂O₅S₂ | 2.769 |
| Compound CE8 | C₄₇H₅₂F₂I₂O₁₁S₂ | 3.222 |

TABLE 6

| Compound | Formula | Ohnishi parameter |
|---|---|---|
| Compound E5 | C₂₆H₁₈F₆O₃S₂ | 2.391 |
| Compound E6 | C₂₇H₂₀F₆O₃S₂ | 2.417 |
| Compound CE9 | C₂₇H₁₆F₆I₂O₅S₂ | 2.636 |
| Compound CE10 | C₂₇H₁₅F₆I₃O₅S₂ | 2.636 |
| Compound CE11 | C₂₉H₁₇F₈I₃O₅S₂ | 2.667 |
| Compound CE12 | C₄₇H₅₀F₆O₉S₂ | 3.000 |

Referring to Tables 4 to 6, since the Ohnishi parameter values of the compounds E1 to E6 are small, it may be confirmed that the etching resistance was improved. In addition, it may be confirmed that the Ohnishi parameter values of the compounds E1 to E6 are smaller than those of compounds CE1 to CE3, and CE5 to CE12.

Embodiments of the present disclosure may provide an organic salt capable of acting as a photoacid generator capable of providing improved sensitivity and/or resolution, and a photoresist composition including the organic salt.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An organic salt represented by Formula 1:
Formula 1 [symbol]
,
wherein, in Formula 1,
L₁ is a single bond or a divalent linking group,
L₁ does not contain oxygen (O),
n1 is an integer of 1 to 5,
R₁ is a cyclic monovalent C₁-C₂₀ hydrocarbon group optionally containing a heteroatom, and
R₁ does not include a group represented by the following Formula R1 or R2, [symbol]
,
and A⁺ is a counter cation.

2. The organic salt of claim 1, wherein
L₁ is a single bond, S, or a linear, branched, or cyclic divalent C₁-C₂₀ hydrocarbon group.

3. The organic salt of claim 1, wherein
L₁ is a single bond, or
L1 is a C₁-C₂₀ alkylene group that is unsubstituted or substituted with deuterium, a halogen, a C₁-C₂₀ alkyl group, or any combination thereof.

4. The organic salt of claim 1, wherein
R₁ is a C₃-C₂₀ cycloalkyl group, a C₁-C₂₀ heterocycloalkyl group, a C₆-C₂₀ aryl group, or a C₃-C₂₀ heteroaryl group, and
R₁ is unsubstituted or substituted with deuterium, a halogen, a C₁-C₂₀ alkyl group, a C₃-C₂₀ cycloalkyl group, a C₆-C₂₀ aryl group, a C₃-C₂₀ heteroaryl group, or any combination thereof.

5. The organic salt of claim 1, wherein
R₁ is a cyclohexyl group, an adamantyl group, a norbornyl group, a tricyclodecanyl group, a tetracyclododecanyl group, a phenyl group, a naphthyl group, or a pyridinyl group, and
R₁ is unsubstituted or substituted with deuterium, a halogen, a C₁-C₂₀ alkyl group, a C₃-C₂₀ cycloalkyl group, a C₆-C₂₀ aryl group, a C₃-C₂₀ heteroaryl group, or any combination thereof.

6. The organic salt of claim 1, wherein
R₁ is a cyclohexyl group, an adamantyl group, a norbornyl group, a tricyclodecanyl group, a tetracyclododecanyl group, a phenyl group, a naphthyl group, or a pyridinyl group, and
R₁ is unsubstituted or substituted with —F, —Cl, —Br, —I, a C₁-C₁₀ alkyl group, a cyclopentyl group, a cyclohexyl group, a phenyl group, a naphthyl group, or any combination thereof.

7. The organic salt of claim 1, wherein A⁺ is a substituted or unsubstituted sulfonium cation, a substituted or unsubstituted iodonium cation, or any combination thereof.

8. The organic salt of claim 1, wherein A⁺ is represented by Formula 2-1 or 2-2: [symbol]
,
wherein in Formulas 2-1 and 2-2,
R₂₁ to R₂₃ are each independently a linear, branched, or cyclic monovalent C₁-C₃₀ hydrocarbon group that optionally contains a heteroatom, and
two adjacent groups of R₂₁ to R₂₃ are optionally bound to each other to form a ring.

9. The organic salt of claim 1, wherein A⁺ is represented by Formula 2-11 or 2-12: [symbol]
,
wherein in Formulas 2-11 and 2-12,
R₂₁ₐ to R₂₁ₑ are each independently a linear, branched, or cyclic monovalent C₁-C₂₀ hydrocarbon group that optionally contains hydrogen, deuterium, a halogen, a cyano group, a hydroxyl group, or a heteroatom,
R₂₂ to R₂₃ are each independently a linear, branched, or cyclic monovalent C₁-C₃₀ hydrocarbon group that optionally contains a heteroatom, and
two adjacent groups of R₂₁ₐ to R₂₁ₑ and R₂₂ to R₂₃ are optionally bound to each other to form a ring.

10. The organic salt of claim 1, wherein A⁺ is represented by one of Formulas 2-21 to 2-23: [symbol]
,
wherein in Formulas 2-21 to 2-23,
R₂₁ₐ to R₂₁ₑ, R₂₂ₐ to R₂₂ₑ, and R₂₃ₐ to R₂₃ₑ are each independently a linear, branched, or cyclic C₁-C₂₀ monovalent hydrocarbon group that optionally contains hydrogen, deuterium, a halogen, a cyano group, a hydroxyl group, or a heteroatom,
two adjacent groups of R₂₁ₐ to R₂₁ₑ, R₂₂ₐ to R₂₃ₑ, and R₂₃ₐ to R₂₃ₑ are optionally bound to each other to form a ring,
b22a and b23a are each an integer of 1 to 4,
A₂₁ and A₂₂ are each independently absent or a benzene ring,
each [symbol] is a carbon-carbon single bond or a carbon-carbon double bond,
L₂₁ is a single bond, O, S, CO, SO, SO₂, CRR′, or NR, and
R and R′ are each independently a linear, branched, or cyclic monovalent C₁-C₂₀ hydrocarbon group that optionally contains hydrogen, deuterium, a halogen, a cyano group, a hydroxyl group, or a heteroatom.

11. The organic salt of claim 1, wherein A⁺ is selected from Group I:
Group I [symbol] [symbol]
.

12. The organic salt of claim 1, wherein the organic salt represented by Formula 1 is represented by Formula 1-1:
Formula 1-1 [symbol]
,
wherein in Formula 1-1,
R₁ and A⁺ are the same as described in Formula 1,
R₁₁ and R₁₂ are each independently hydrogen, a halogen, a C₁-C₂₀ alkyl group, or a C₁-C₂₀ halogenated alkyl group, and n11 is an integer of 0 to 5.

13. The organic salt of claim 1, wherein the organic salt represented by Formula 1 is selected from Group II:
Group II [symbol]
,
wherein in Group II, A⁺ is a counter cation.

14. A photoresist composition, comprising:
the organic salt of claim 1;
an organic solvent; and a base resin.

15. The photoresist composition of claim 14, wherein
the organic salt is a photo-decomposable compound that is decomposed by exposure to light.

16. The photoresist composition of claim 15, further comprising:
a quencher.

17. The photoresist composition of claim 14, wherein the base resin comprises a repeating unit represented by Formula 4:
Formula 4 [symbol]
,
wherein in Formula 4,
R₄₁ is hydrogen, deuterium, a halogen, a linear or branched C₁-C₂₀ alkyl group, a linear or branched C₁-C₂₀ halogenated alkyl group,
L₄₁ is a single bond, O, C(═O), C(═O)O, OC(═O), C(═O)NH, NHC(═O), a substituted or unsubstituted C₁-C₃₀ alkylene group, a substituted or unsubstituted C₃-C₃₀ cycloalkylene group, a substituted or unsubstituted C₃-C₃₀ heterocycloalkylene group, a substituted or unsubstituted C₂-C₃₀ alkenylene group, a substituted or unsubstituted C₃-C₃₀ cycloalkenylene group, a substituted or unsubstituted C₃-C₃₀ heterocycloalkenylene group, a substituted or unsubstituted C₆-C₃₀ arylene group, or a substituted or unsubstituted C₁-C₃₀ heteroarylene group,
a41 is an integer of 1 to 6,
X₄₁ is an acid labile group,
* and *′ are each a binding site with a neighboring atom, and
if L₄₁ is substituted, a substituent of L₄₁ is
deuterium, —F, —Cl, —Br, —I, —CD₃, —CD₂H, —CDH₂, —CF₃, —CF₂H, —CFH₂, a hydroxyl group, a carboxylic acid group or a salt thereof, a sulfonic acid group or a salt thereof, a phosphate group or a salt thereof, a C₁-C₂₀ alkyl group, a C₂-C₂₀ alkenyl group, a C₂-C₂₀ alkynyl group, or a C₁-C₂₀ alkoxy group,
a C₁-C₂₀ alkyl group, a C₂-C₂₀ alkenyl group, a C₂-C₂₀ alkynyl group, or a C₁-C₂₀ alkoxy group, substituted with deuterium, —F, —Cl, —Br, —I, —CD₃, —CD₂H, —CDH₂, —CF₃, —CF₂H, —CFH₂, a hydroxyl group, a carboxylic acid group or a salt thereof, a sulfonic acid group or a salt thereof, a phosphate group or a salt thereof, a C₃-C₁₀ cycloalkyl group, a C₁-C₁₀ heterocycloalkyl group, a C₃-C₁₀ cycloalkenyl group, or a C₁-C₁₀ heterocycloalkenyl group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, a C₁-C₂₀ heteroarylthio group, a monovalent non-aromatic condensed polycyclic group, a monovalent non-aromatic hetero-condensed polycyclic group, or any combination thereof,
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, a C₁-C₂₀ heteroarylthio group, a monovalent non-aromatic condensed polycyclic group, or a monovalent non-aromatic hetero-condensed polycyclic group,
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₂₀ aryl group, a C₆-C₂₀ aryloxy group, a C₆-C₂₀ arylthio group, a C₁-C₂₀ heteroaryl group, a monovalent non-aromatic condensed polycyclic group, or a monovalent non-aromatic hetero-condensed polycyclic group, substituted with deuterium, —F, —Cl, —Br, —I, —CD₃, —CD₂H, —CDH₂, —CF₃, —CF₂H, —CFH₂, a hydroxyl group, a carboxylic acid group or a salt thereof, a sulfonic acid group or a salt thereof, a phosphate group or a salt thereof, a C₁-C₂₀ alkyl group, a C₂-C₂₀ alkenyl group, a C₂-C₂₀ alkynyl group, a C₁-C₂₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₂₀ aryl group, a C₆-C₂₀ aryloxy group, a C₆-C₂₀ arylthio group, a C1-C60 heteroaryl group, a C₁-C₂₀ heteroaryloxy group, a C₁-C₂₀ heteroarylthio group, a monovalent non-aromatic condensed polycyclic group, a monovalent non-aromatic hetero-condensed polycyclic group, or any combination thereof.

18. The photoresist composition of claim 14, wherein the organic solvent comprises an alcohol-based solvent, an ether-based solvent, a ketone-based solvent, an amide-based solvent, an ester-based solvent, a sulfoxide-based solvent, a hydrocarbon-based solvent, or any combination thereof.

19. A method of forming a pattern, comprising:
forming a photoresist film by applying the photoresist composition of claim 14 onto a substrate;
exposing at least a portion of the photoresist film with high-energy rays; and
developing the exposed photoresist film by using a developing solution.

20. The method of claim 19, wherein the exposing is performed by irradiation with at least one of ultraviolet (UV) rays, deep ultraviolet (DUV) rays, extreme ultraviolet (EUV) rays, or electron beams (EBs).
